(19) United States
(12) Patent Application Publication
(10) Pub. No.: US 2025/0375620 A1
(43) Pub. Date: Dec. 11, 2025

(54) THERAPEUTIC LIGHT SYSTEM

(71) Applicant: Dahlia Holdings, LLC, Urbandale, IA (US)

(72) Inventors: Steve Blumenschein, Van Meter, IA (US); Tim Mohrfeld, III, Van Meter, IA (US); Joseph E. Van Riper, III, Germantown, MD (US); David Mike Girton, Frederick, MD (US); Kory Pohlman, West Des Moines, IA (US); Jill Heathcote, Van Meter, IA (US)

(21) Appl. No.: 19/301,545

(22) Filed: Aug. 15, 2025

Related U.S. Application Data
(63) Continuation-in-part of application No. 18/975,035, filed on Dec. 10, 2024.
(60) Provisional application No. 63/610,623, filed on Dec. 15, 2023.
(60) Provisional application No. 63/684,521, filed on Aug. 19, 2024.
(60) Provisional application No. 63/684,521, filed on Aug. 19, 2024.
(60) Provisional application No. 63/711,921, filed on Oct. 25, 2024.

(51) Int. Cl. A61N 5/06

(52) U.S. Cl. CPC A61N 5/0616; A61N 2005/0626; A61N 2005/0638; A61N 2005/0652; A61N 2005/0659; A61N 2005/0663; A61N 2005/0666

(57) ABSTRACT

In one or more arrangements, a system is presented for therapeutic light treatment. In one or more arrangements, the system includes chamber having a bed and a canopy operably connected thereto. The system includes a plurality of flexible light array pads positioned in the chamber so as to expose the patient to therapeutic light when operated. In one or more arrangements, the flexible light array pads have a plurality of light emitters positioned respectively in a plurality of windows. In one or more arrangements, the plurality of light emitters are configured to emit light in a plurality of different wavelengths. In one or more arrangements, the system includes a control system configured to control wavelength, intensity, waveforms and/or patterns of therapeutic light generated by the plurality of light emitters.

### RELATED APPLICATIONS

This application claims priority to U.S. Provisional Patent Application No. 63/684,521, filed Aug. 19, 2024 and titled “THERAPEUTIC LIGHT SYSTEM”, which is hereby fully incorporated by reference herein. This application is also a continuation-in-part of U.S. Utility patent application Ser. No. 18/975,035, filed on Dec. 10, 2024 and titled “FLEXIBLE LIGHT THERAPY PAD WITH REFLECTOR INSERTS”, which claims priority to: U.S. Provisional Patent Application No. 63/610,623, filed Dec. 15, 2023 and titled “THERAPEUTIC LIGHT SYSTEM”; U.S. Provisional Patent Application No. 63/684,521, filed Aug. 19, 2024 and titled “THERAPEUTIC LIGHT SYSTEM”; and U.S. Provisional Patent Application No. 63/711,921, filed Oct. 25, 2024 and titled “FLEXIBLE LIGHT THERAPY PAD WITH REFLECTOR INSERTS”, each of the foregoing being hereby fully incorporated by reference herein.

### FIELD OF THE DISCLOSURE

The disclosure generally relates to light therapy and more specifically to systems and methods for application of light therapy.

### OVERVIEW OF THE DISCLOSURE

Light therapy has been shown an ability to provide several different benefits depending on the application. Stimulating the body's natural processes with red light therapy is safe and has no known side effects. Red light therapy has gone through extensive clinical research and test results show overwhelmingly positive results for skin health, collagen production, physical performance & muscle recovery, sleep, joint pain, inflammation, and much more. For example, red light therapy can result in fat loss and fat cell reduction. Red light triggers the formation of small openings or pores in fat cells, which releases fatty acids known as lipids. One study found that just four minutes of exposure to 635 nm red light caused 80% of lipids to be released from fat cells, and by six minutes, almost all of the fat had been released.

Visible red light is capable of penetrating the skin to a depth of about 8 To 10 mm. Once absorbed, the light energy is converted to cellular energy, stimulating the body's natural processes on a cellular level and kicking off a whole series of metabolic events, which may include but are not limited to, for example,: increased circulation and the formation of new capillaries, increase lymph system activity; increase production of collagen and fibroblasts; increased release of raw cellular energy; increase cellular clean up; tissue granulation simulated; and/or reduced inflammation. All of these things work together to produce many benefits for you in the areas of anti-aging, the healing of injuries, and/or the relief of pain, among other benefits.

Through careful observation it has been surprisingly discovered that targeted application of infrared light and/or near infrared light can supplement and improve the results and/or experience of red light therapy. For ease of reference, light in the spectral ranges of red light, near infrared light, and infrared light may be jointly or individually be referred to generally as therapeutic light.

However, therapeutic light treatment presents many challenges. For example, it can be difficult to generate and direct therapeutic light with generally equal intensity across a target treatment area of a patient. For example, some application systems utilize a chamber (e.g., a bed and/or booth) that surrounds the patient with therapeutic lights. However, due to the duration of treatments, is it more comfortable for patients to be in a seated or lying position, which positions therapeutic lights under the patient in closer proximity to the patient's skin. In such a position, therapeutic lights of some red light therapy systems may not apply therapeutic light evenly. Furthermore, the close proximity of the patient to such therapeutic lights may cause buildup of heat, particularly when near infrared light and/or infrared light are used, which may cause discomfort or harm to the patient.

Therefore, for all the reasons stated above, and the reasons stated below, there is a need in the art for an improved system and method for therapeutic light treatment.

Thus, it is a primary objective of the disclosure to provide a system and method for therapeutic light treatment that improves upon the state of the art.

Another objective of the disclosure is to provide a system and method for therapeutic light treatment that applies therapeutic light evenly to a target area.

Yet another objective of the disclosure is to provide a system and method for therapeutic light treatment that can apply a plurality of types of therapeutic light.

Another objective of the disclosure is to provide a system and method for therapeutic light treatment that can apply different prescriptions of therapeutic light patterns for various different treatments.

Yet another objective of the disclosure is to provide a system and method for therapeutic light treatment that reduces patient risk.

Another objective of the disclosure is to provide a system and method for therapeutic light treatment that is sanitary.

Yet another objective of the disclosure is to provide a system and method for therapeutic light treatment that is effective.

Another objective of the disclosure is to provide a system and method for therapeutic light treatment that is reliable.

Yet another objective of the disclosure is to provide a system and method for therapeutic light treatment that has a long useful life.

Another objective of the disclosure is to provide a system and method for therapeutic light treatment that is easy to use.

These and other objects, features, or advantages of the disclosure will become apparent from the specification, figures, and claims.

### SUMMARY

In one or more arrangements, a system is presented for therapeutic light treatment. In one or more arrangements, the system includes a chamber having a bed and a canopy operably connected thereto. The system includes a plurality of flexible light array pads positioned in the chamber so as to expose the patient to therapeutic light when operated. In one or more arrangements, the flexible light array pads have a plurality of light emitters positioned respectively in a plurality of windows. In one or more arrangements, the plurality of light emitters are configured to emit light in a plurality of different wavelengths. In one or more arrangements, the system includes a control system configured to control therapeutic light generated by the plurality of light emitters according to a prescription selected by the user. In one or more arrangements, the prescription specifies wavelength, intensity, duration, waveforms, and/or patterns of the light to be emitted by the plurality of flexible light array pads. In one or more arrangements, the system is configured to measure the temperature of the plurality of flexible light array pads and automatically adjust operation of light emitters based on the measured temperature.

In one or more arrangements, the flexible light array pads have an upper surface and a lower surface extending between a forward edge, a rearward edge, and opposing side edges. In one or more arrangements, the flexible light array pads have a plurality of windows formed in the upper surface and a plurality of light emitters positioned respectively in the plurality of windows. In one or more arrangements, the plurality of light emitters are configured to emit light in a plurality of different spectra. In one or more arrangements, the flexible light array pads have reflective inserts positioned within the windows that are configured to direct therapeutic light produced by light emitters out through the plurality of windows.

### BRIEF DESCRIPTION OF THE FIGURES

FIG. 1 shows a top side view of a flexible light array, in accordance with one or more arrangements.

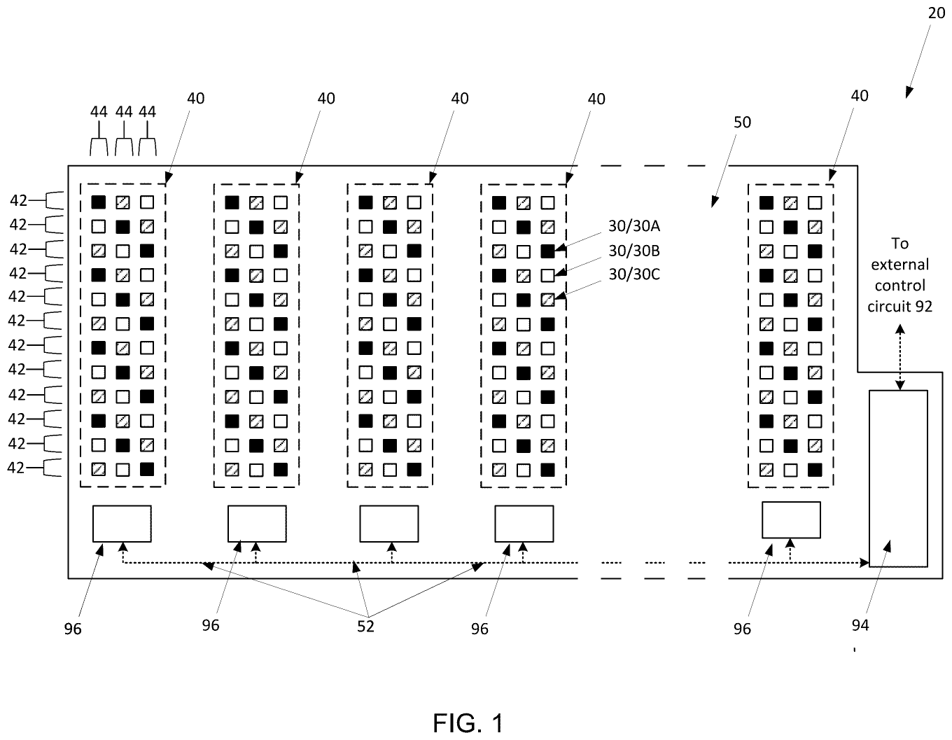

FIG. 2 shows a front cross sectional view of a flexible light array, in accordance with one or more arrangements.

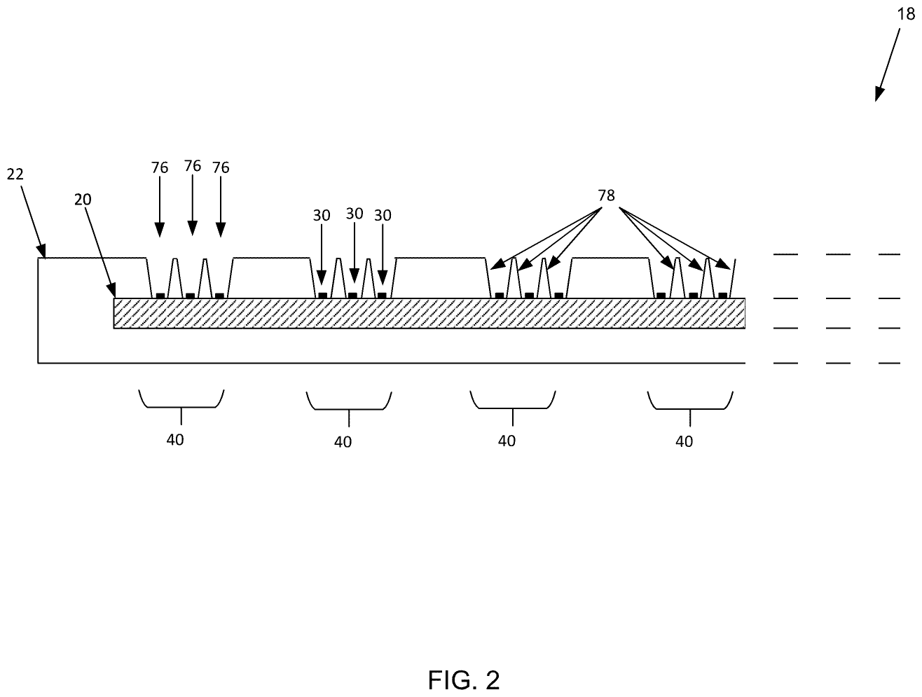

FIG. 3 is a right side view of the light therapy bed; the view showing a canopy of the bed in the lowered position.

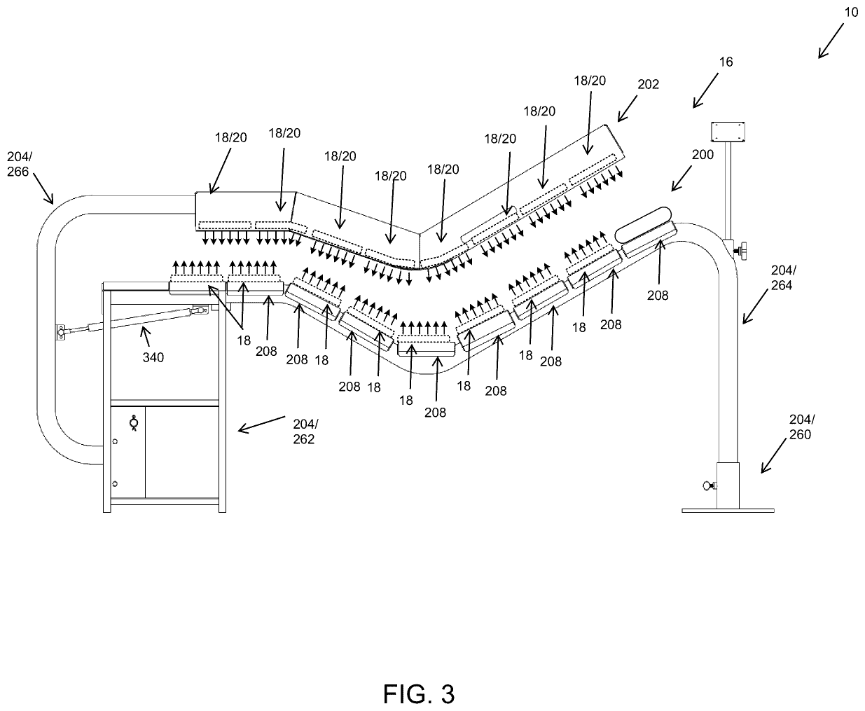

FIG. 4 is an upper front right perspective view of a light therapy bed; the view showing a canopy of the bed in a lowered position.

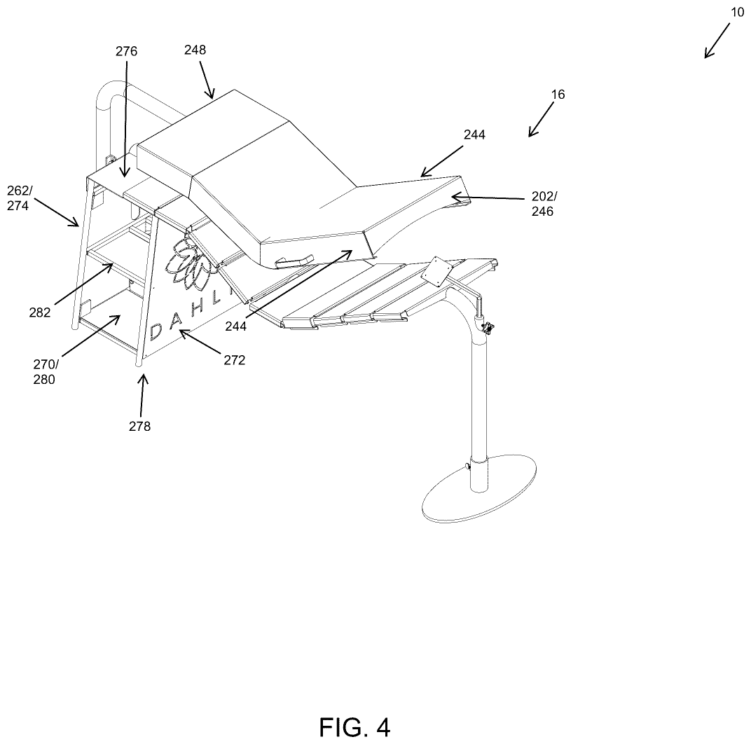

FIG. 5 is an upper front right perspective view of the light therapy bed; the view showing the canopy of the bed in a raised position.

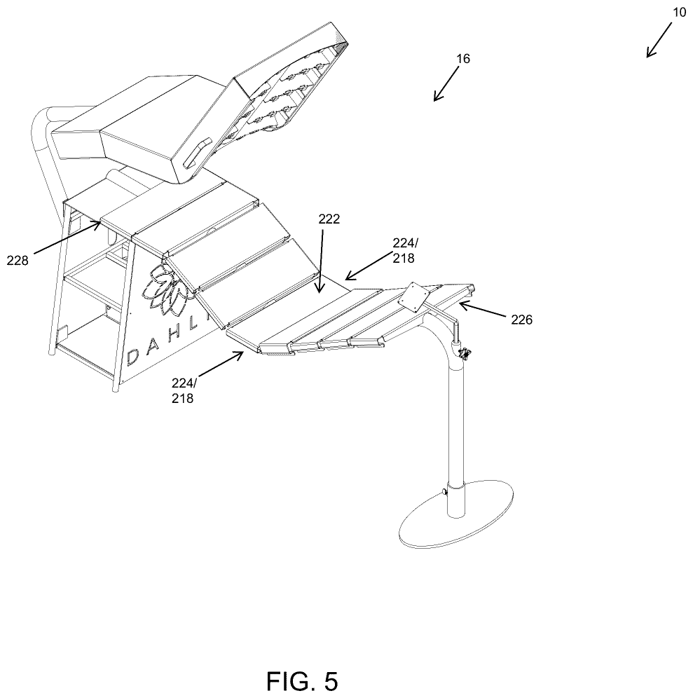

FIG. 6 is a right side view of the light therapy bed; the view showing a canopy of the bed in the lowered position.

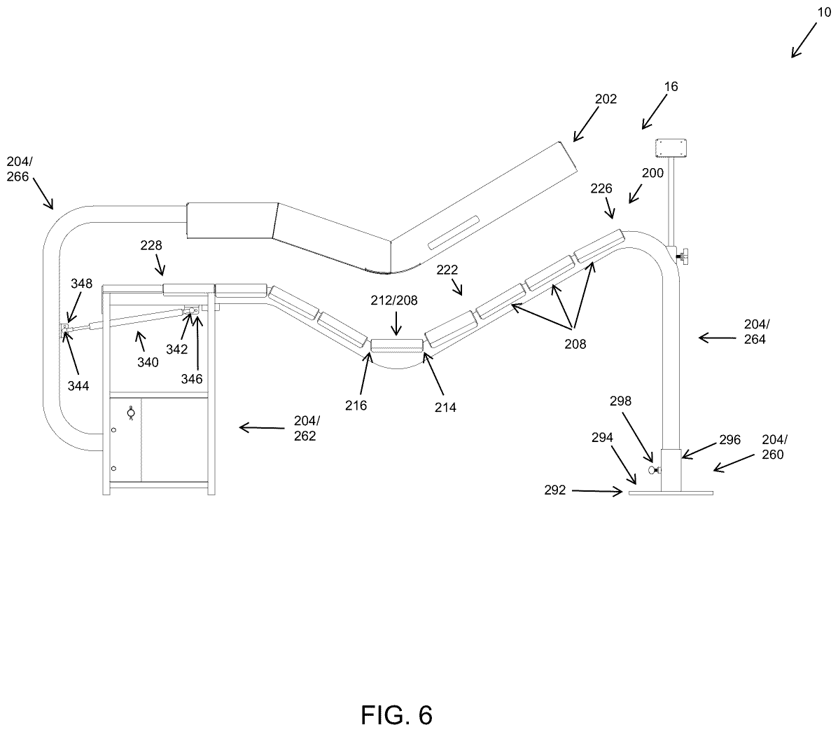

FIG. 7 is a right side view of the light therapy bed; the view showing the canopy of the bed in the raised position.

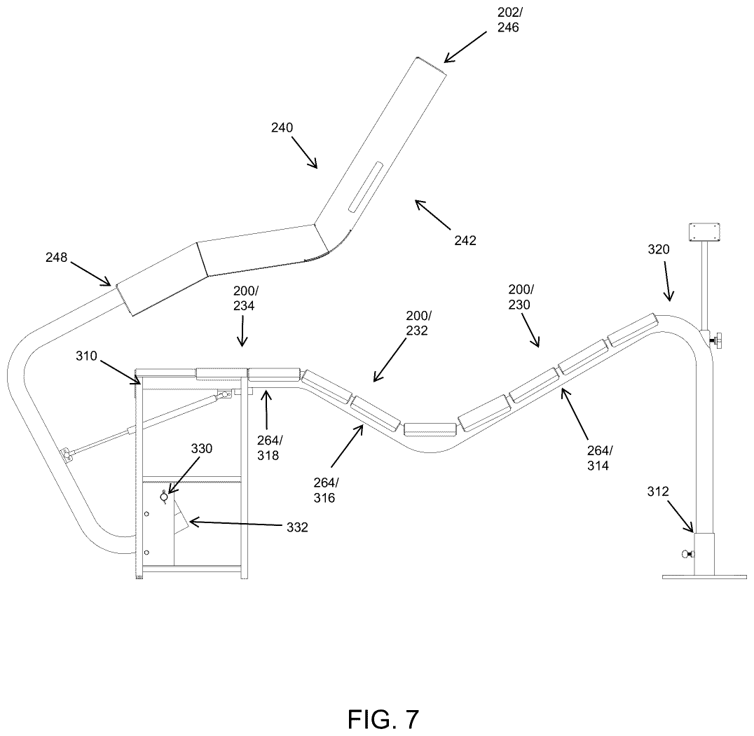

FIG. 8 is a top view of the light therapy bed; the view showing the canopy of the bed in the lowered position.

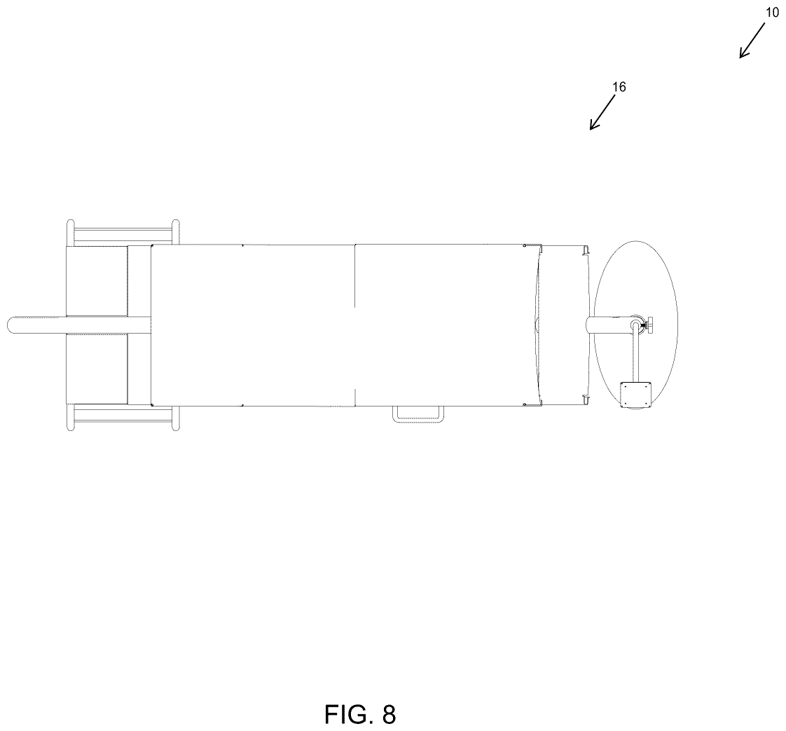

FIG. 9 is a top view of a light therapy bed; the view showing the canopy of the bed in a raised position.

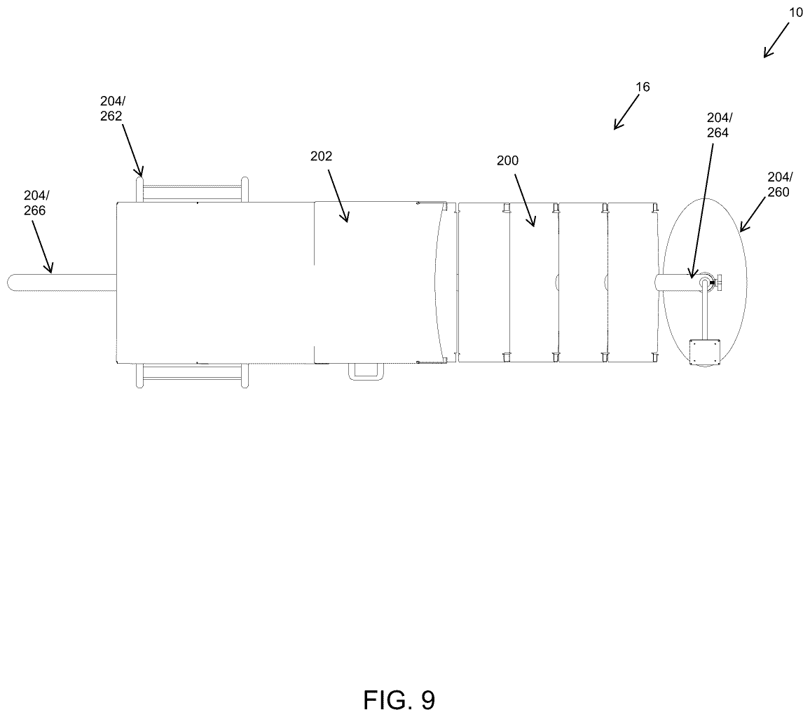

FIG. 10 is a front view of the light therapy bed; the view showing the canopy of the bed in the lowered position.

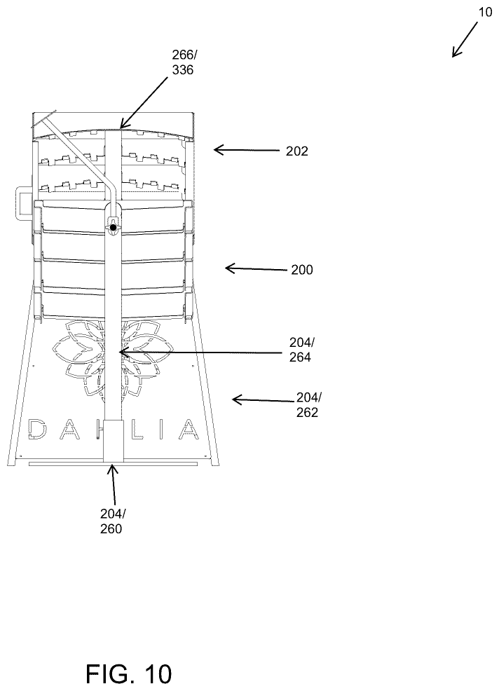

FIG. 11 is a front view of the light therapy bed; the view showing the canopy of the bed in the raised position.

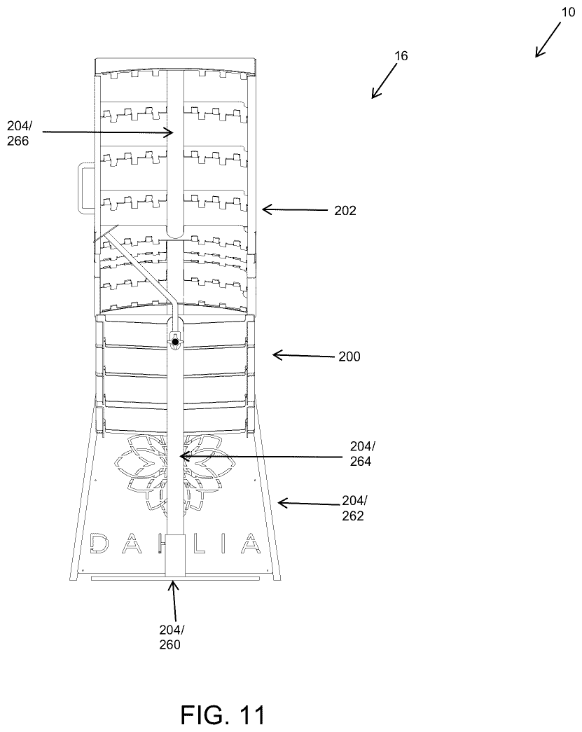

FIG. 12 is a rear view of the light therapy bed; the view showing the canopy of the bed in the lowered position.

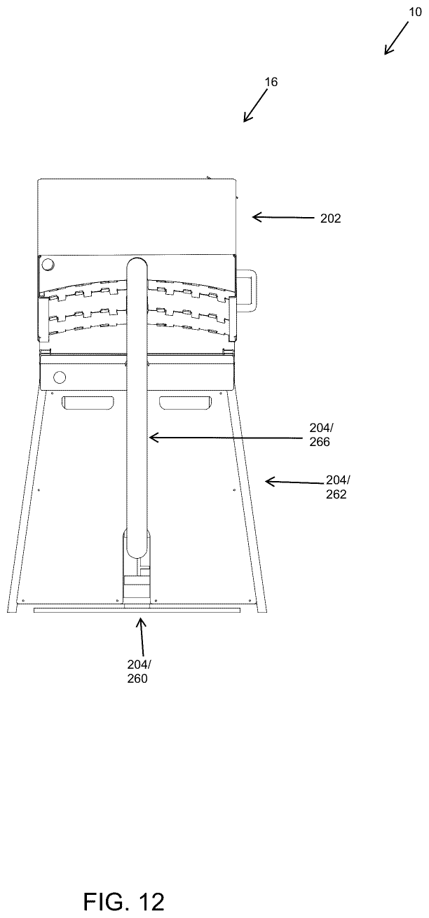

FIG. 13 is a bottom view of the light therapy bed; the view showing the canopy of the bed in the lowered position.

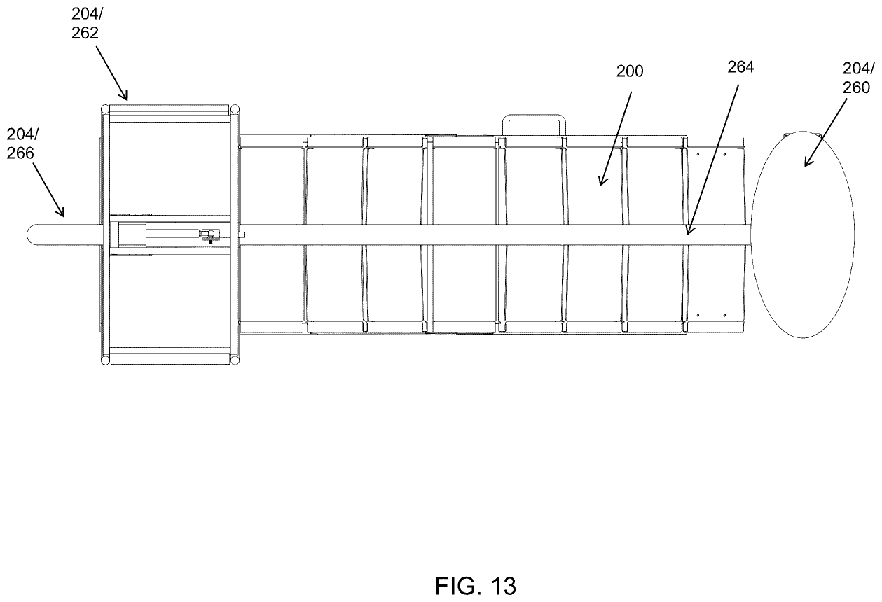

FIG. 14 shows a block level diagram of an example control system for control of a plurality of flexible light arrays, in accordance with one or more arrangements.

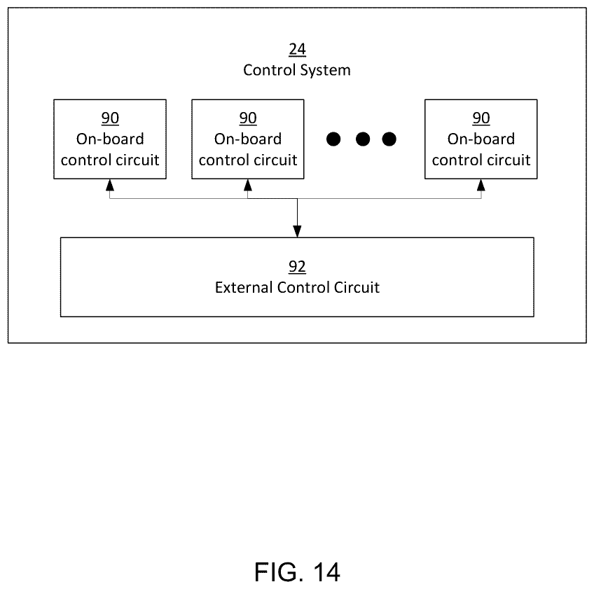

FIG. 15 shows a block level diagram of an example control system for controlling a flexible light array, in accordance with one or more arrangements.

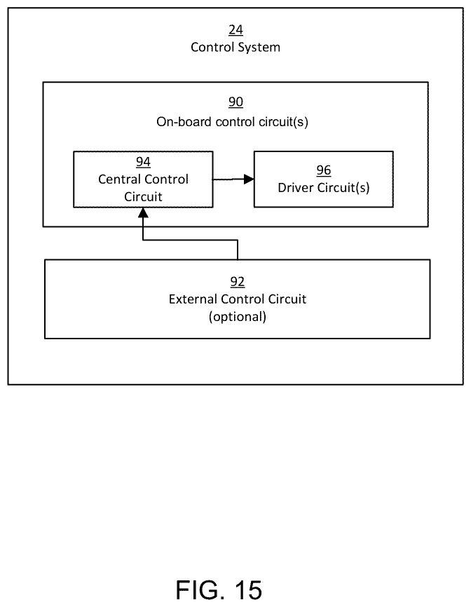

FIG. 16 shows a block level diagram of an example implementation of a processing system that may be utilized for implementation of on-board control circuit, external control circuit, control system generally, and/or various other components of system, in accordance with one or more arrangements.

FIG. 17 shows a flowchart of an example process for controlling operation of light emitters of a flexible light array to generate therapeutic light for a selected prescription treatment, in accordance with one or more arrangements.

FIG. 18 shows a flowchart of an example process for determining an amount of power for a particular prescription treatment in performance of the example process shown in FIG. 17, in accordance with one or more arrangements.

FIG. 19 shows a flowchart of an example subprocess for adjusting power for light emitters based on temperature readings of sensors in performance of the process shown in FIG. 17, in accordance with one or more arrangements.

FIG. 20 shows a top side view of a flexible light array pad for a therapeutic light treatment system, in accordance with one or more arrangements.

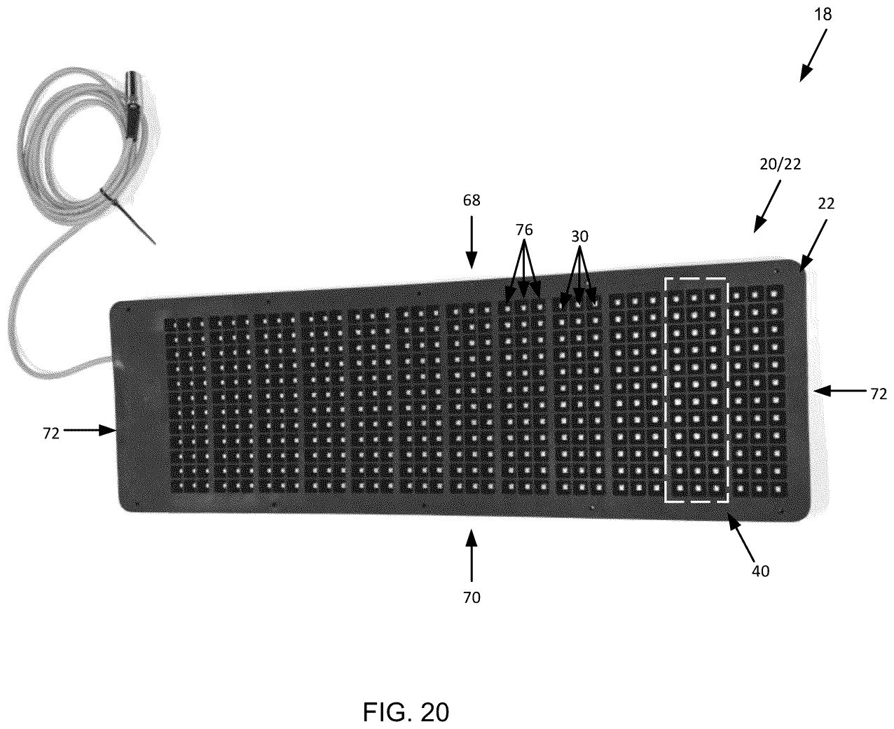

FIG. 21 shows a front view of a flexible light array pad for a therapeutic light treatment system, in accordance with one or more arrangements.

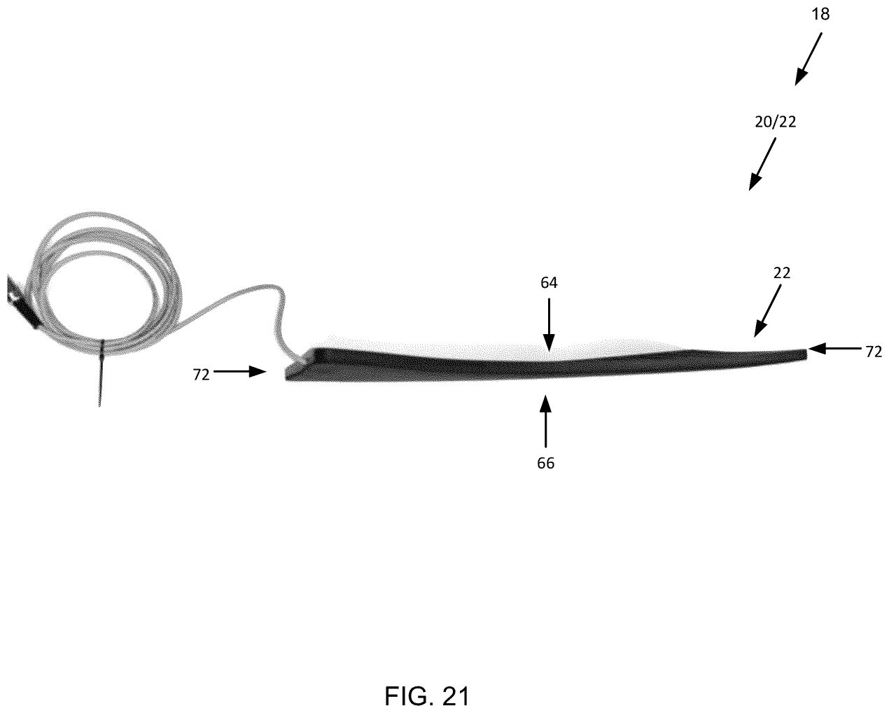

FIG. 22 shows a front view of a flexible light array pad for a therapeutic light treatment system, in accordance with one or more arrangements.

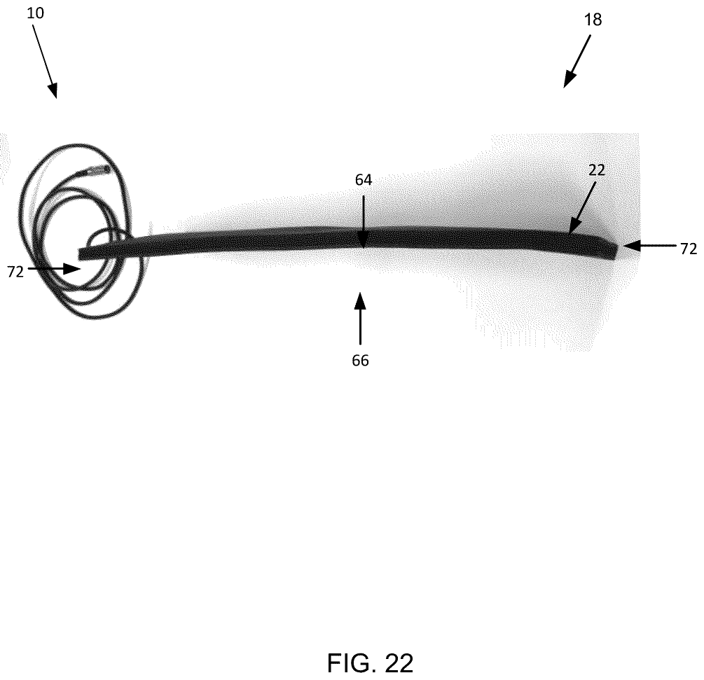

FIG. 23 shows a bottom view of a flexible light array pad for a therapeutic light treatment system, in accordance with one or more arrangements.

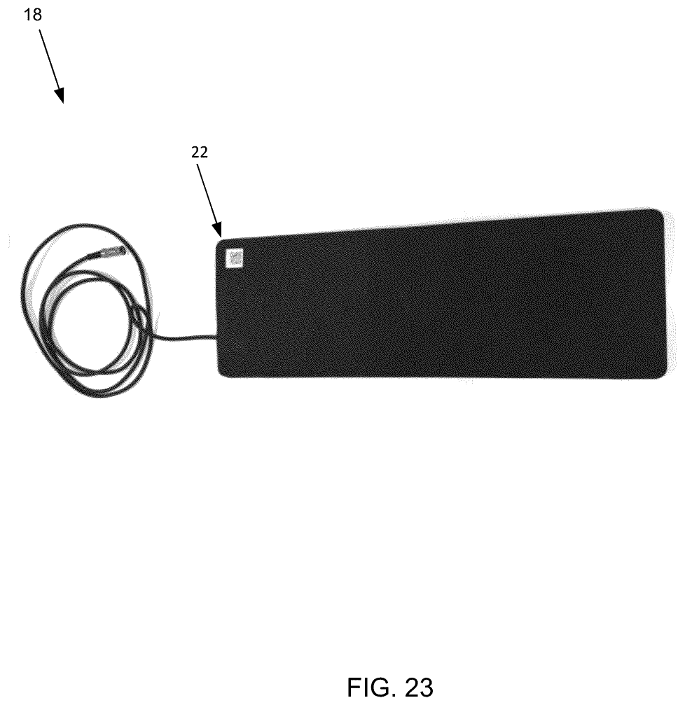

FIG. 24 shows a top side perspective view of a flexible light array pad for a therapeutic light treatment system, in accordance with one or more arrangements.

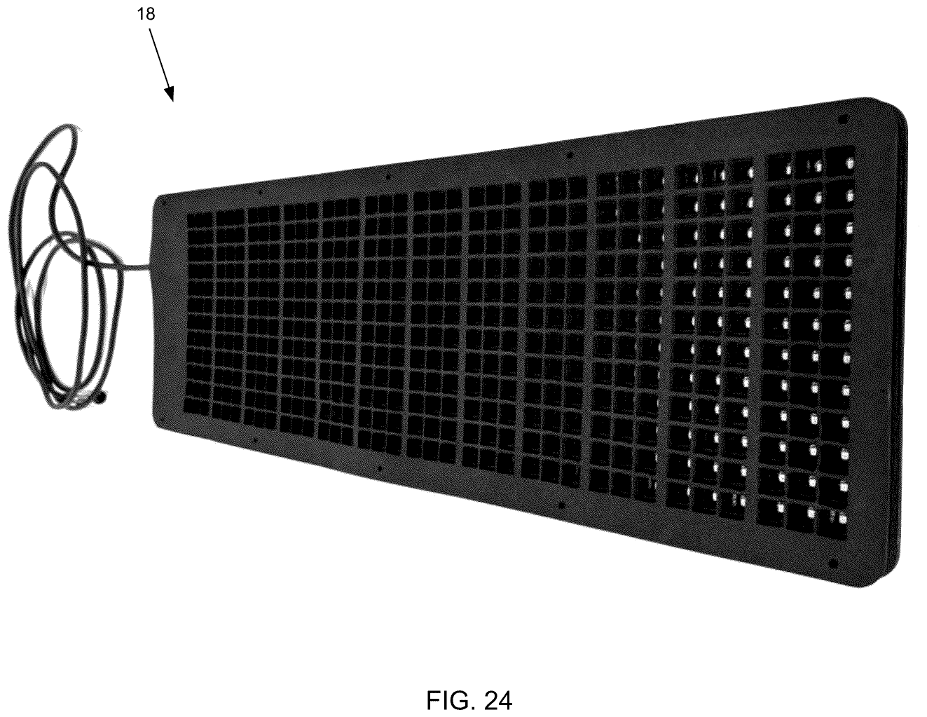

FIG. 25 shows a top side perspective view of a flexible light array pad for a therapeutic light treatment system, in accordance with one or more arrangements.

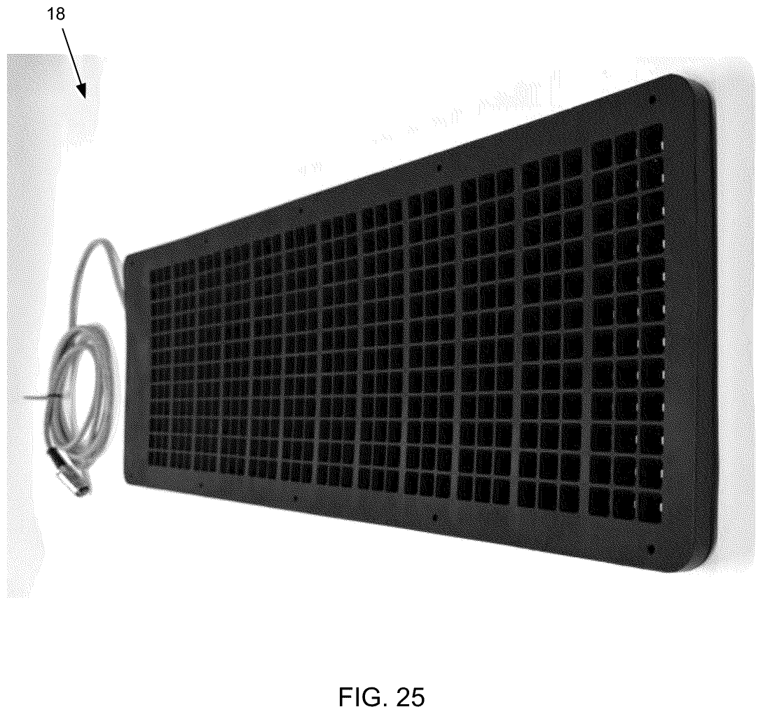

FIG. 26 shows a partial top side view of a flexible light array pad for a therapeutic light treatment system, in accordance with one or more arrangements; the view showing reflective inserts positioned within some windows of the flexible light array pad.

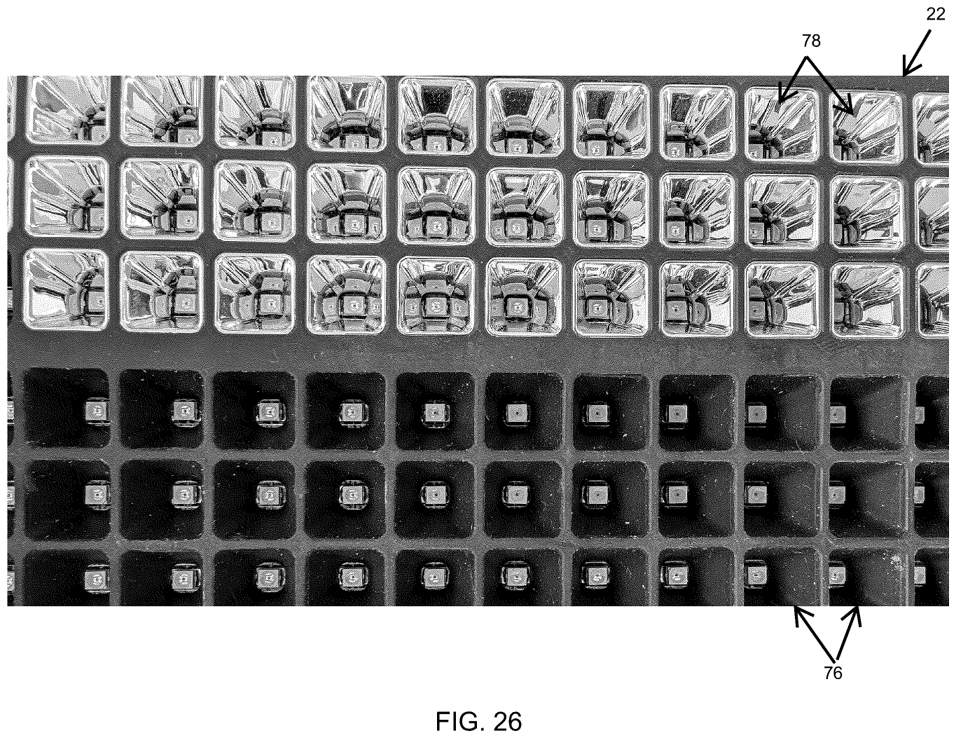

FIG. 27 shows a partial perspective view of a flexible light array pad for a therapeutic light treatment system, in accordance with one or more arrangements; the view showing reflective inserts positioned within some windows of the flexible light array pad.

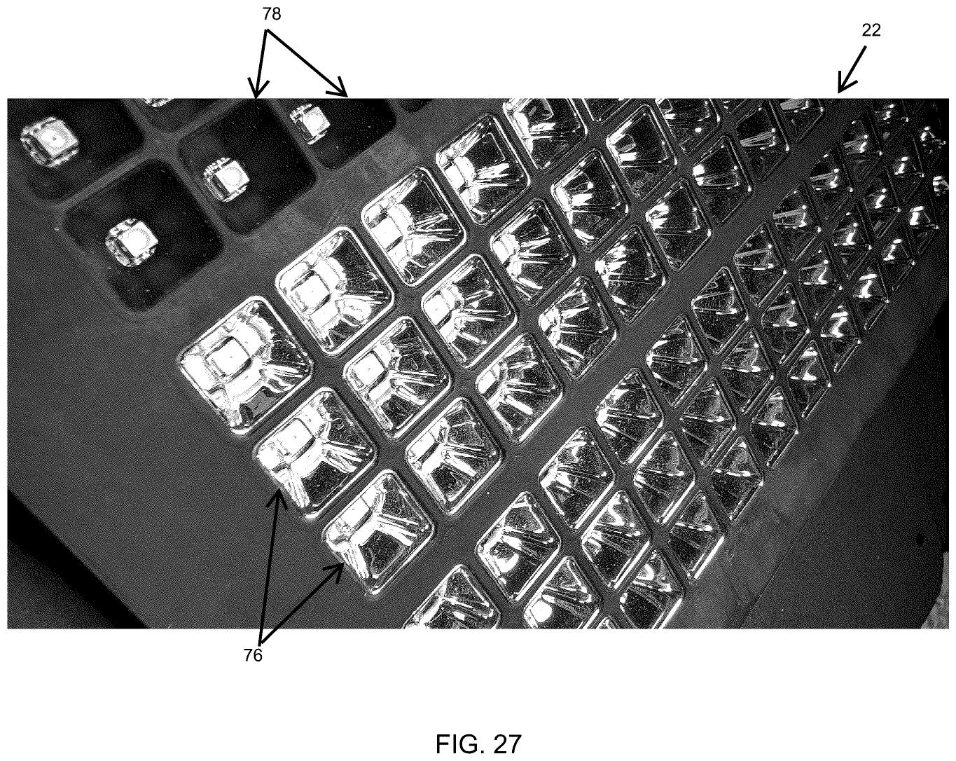

FIG. 28 shows an upper perspective view of a reflective insert for a flexible light array pad, in accordance with one or more arrangements.

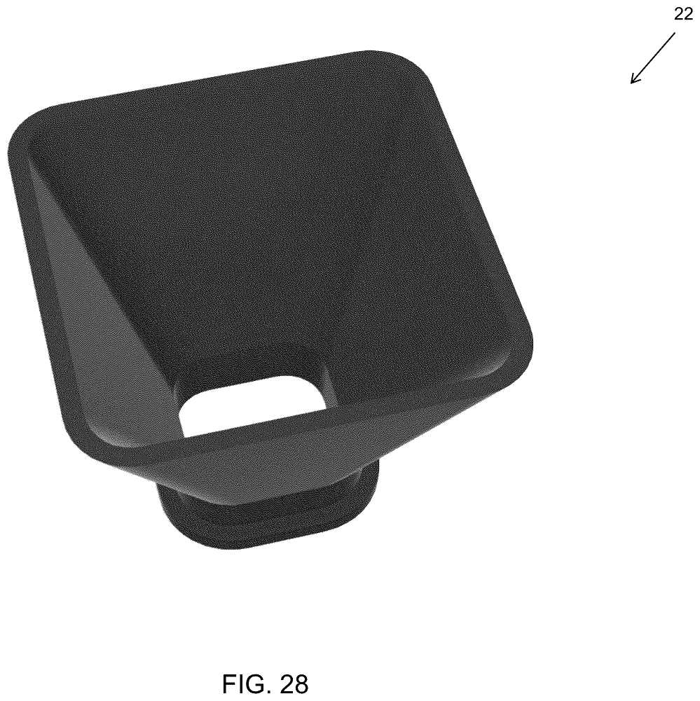

FIG. 29 shows an upper perspective view of a reflective insert for a flexible light array pad, in accordance with one or more arrangements.

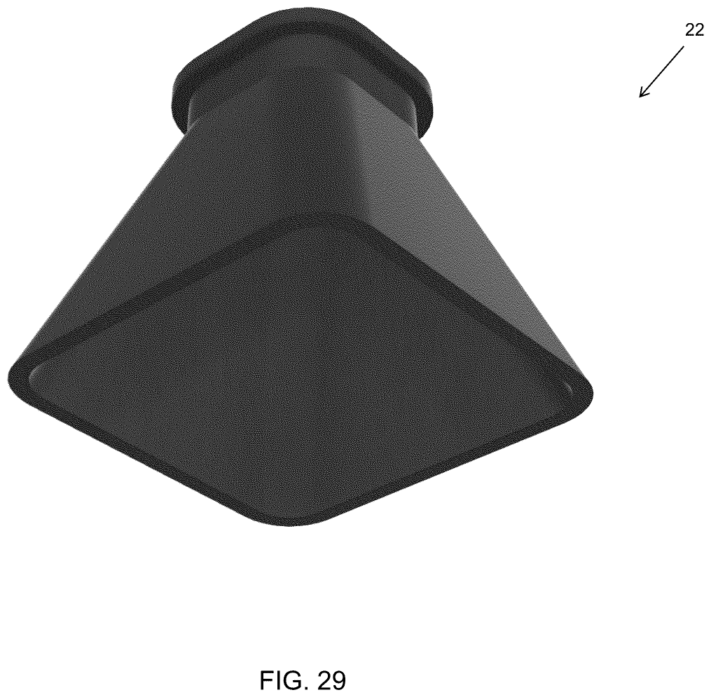

FIG. 30 shows a lower perspective view of a reflective insert for a flexible light array pad, in accordance with one or more arrangements.

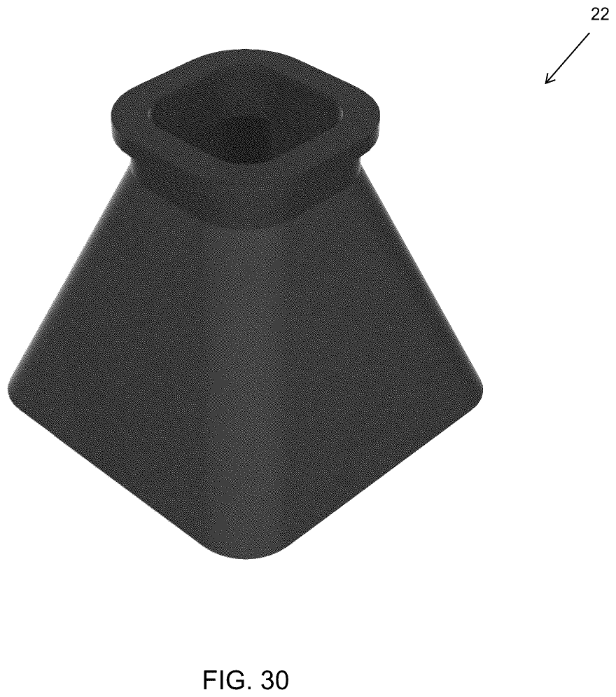

FIG. 31 shows a side view of a reflective insert for a flexible light array pad, in accordance with one or more arrangements.

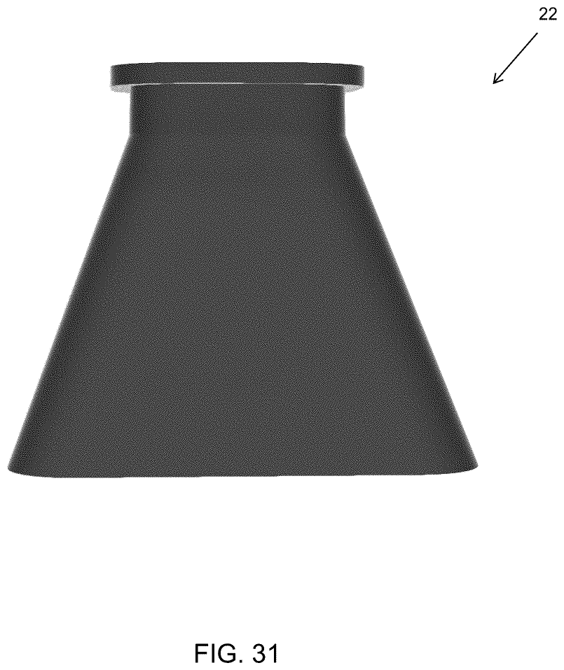

FIG. 32 shows a top view of a reflective insert for a flexible light array pad, in accordance with one or more arrangements.

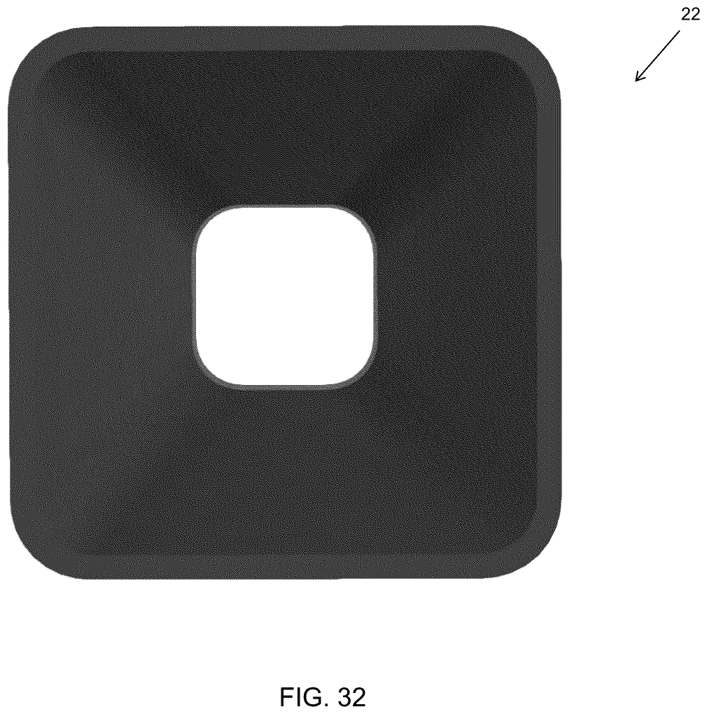

FIG. 33 shows a bottom view of a reflective insert for a flexible light array pad, in accordance with one or more arrangements.

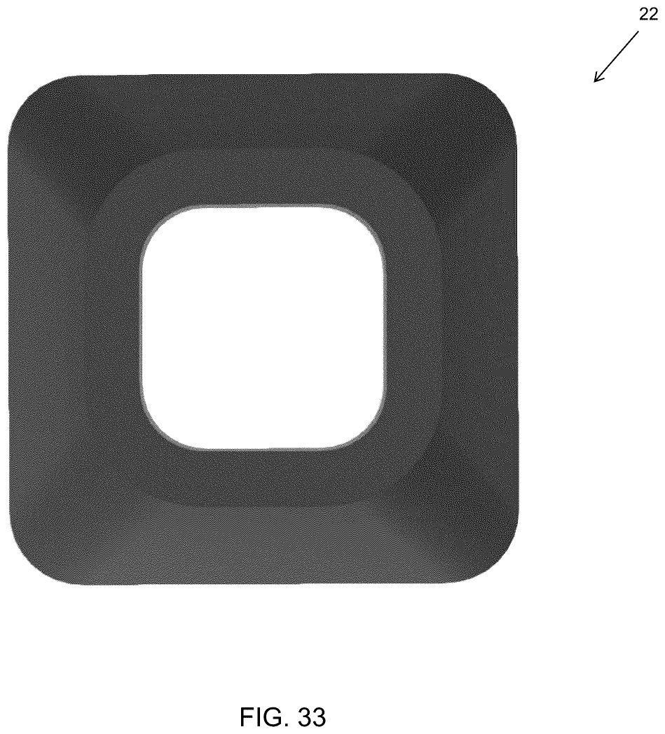

FIG. 34 shows an upper perspective view of a reflective insert for a flexible light array pad, in accordance with one or more arrangements.

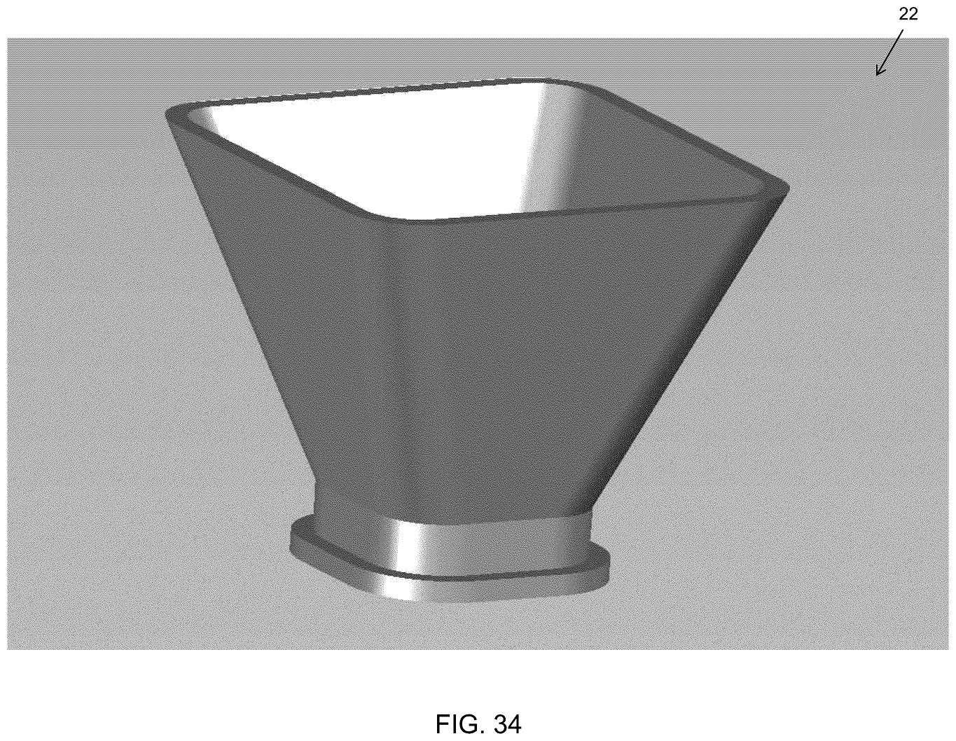

FIG. 35 shows an upper perspective view of a reflective insert for a flexible light array pad, in accordance with one or more arrangements.

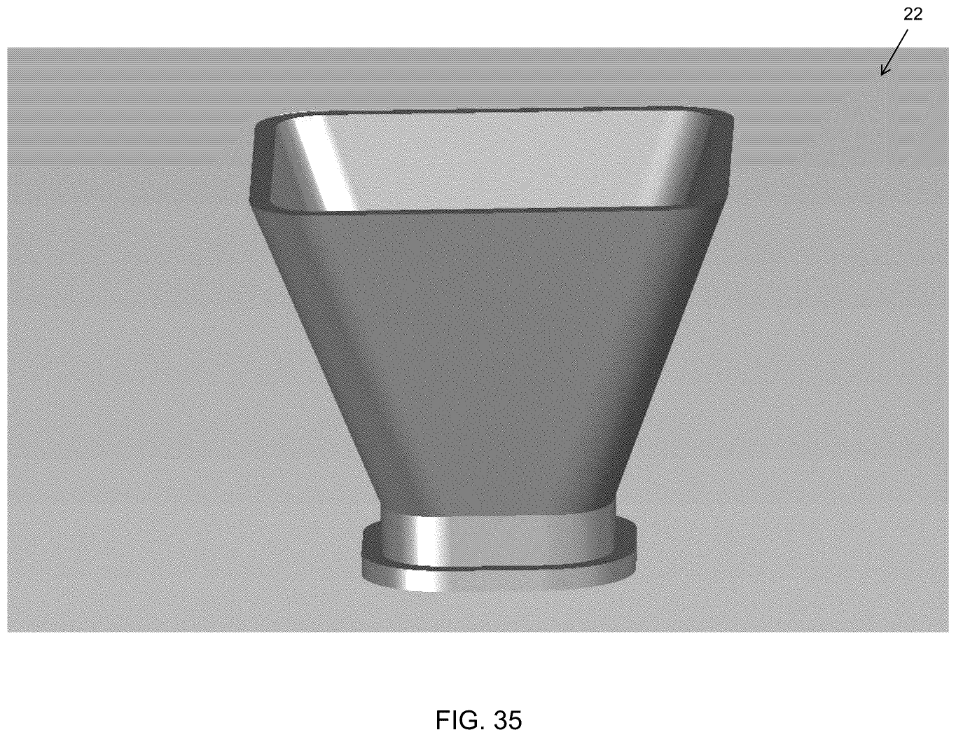

FIG. 36 shows a top view of a reflective insert for a flexible light array pad, in accordance with one or more arrangements.

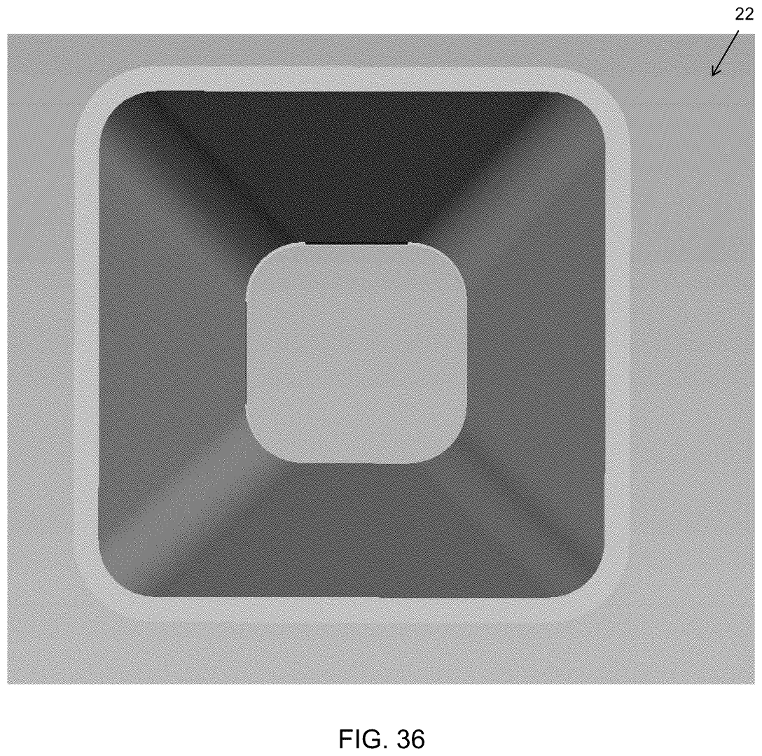

FIG. 37 shows a bottom view of a reflective insert for a flexible light array pad, in accordance with one or more arrangements.

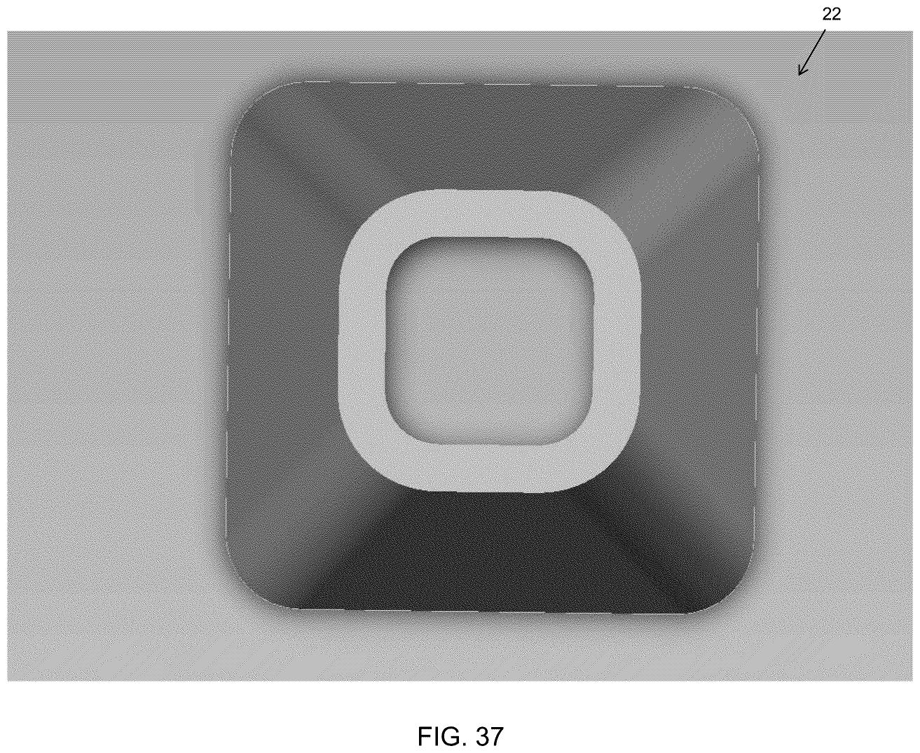

FIG. 38 shows a top view of a flexible light array, in accordance with one or more arrangements.

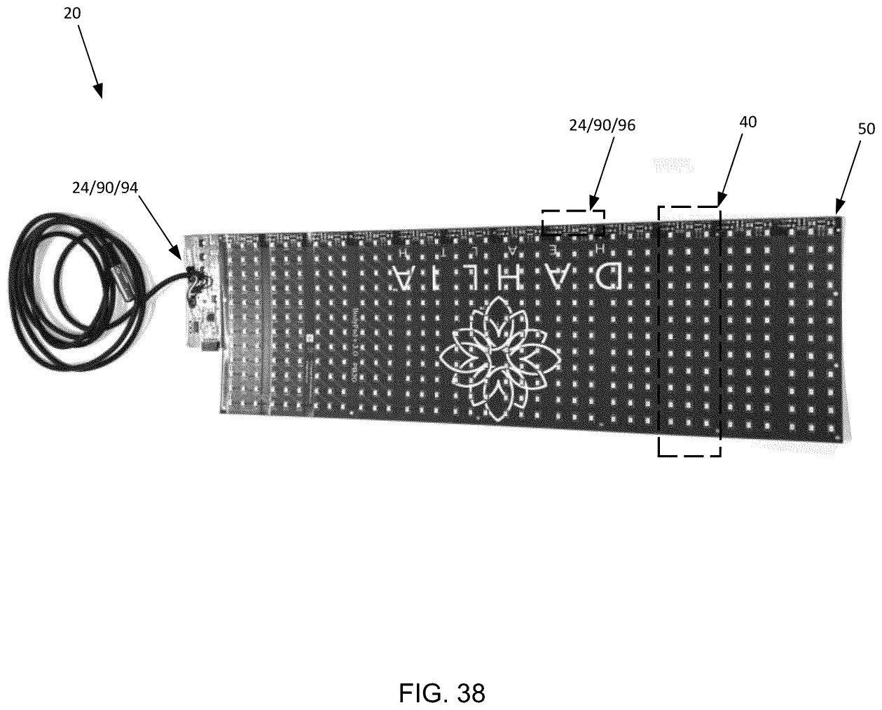

FIG. 39 shows a bottom view of a flexible light array, in accordance with one or more arrangements.

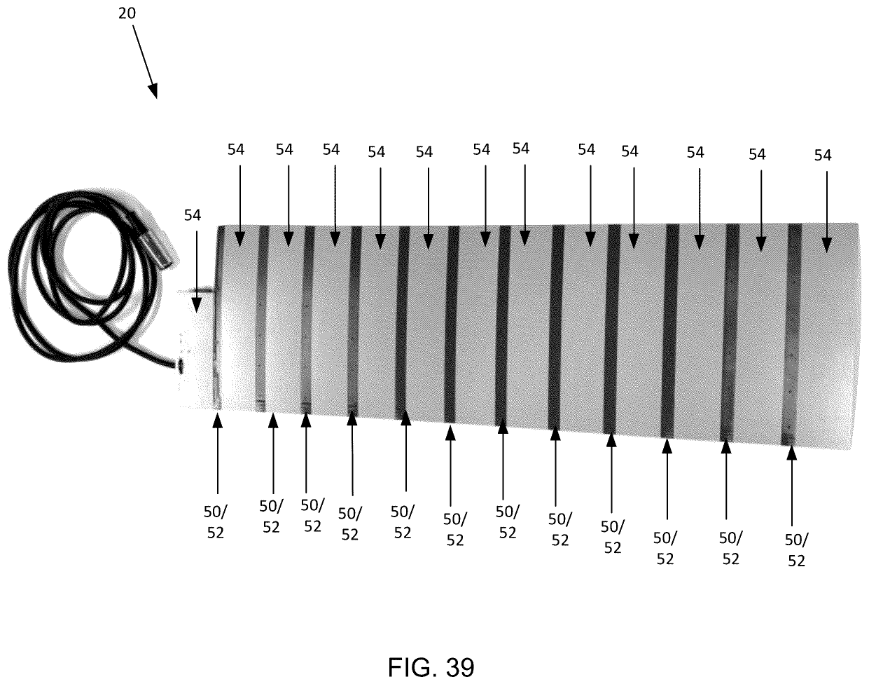

FIG. 40 shows a top side perspective view of a flexible light array, in accordance with one or more arrangements.

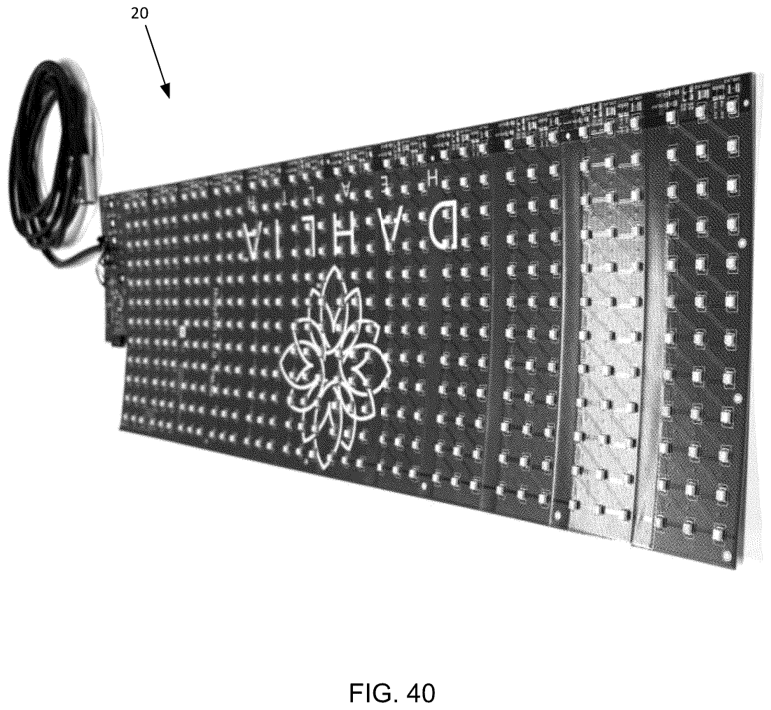

FIG. 41 shows a perspective view of an external control circuit configured to control operation of one or more flexible light arrays, in accordance with one or more arrangements.

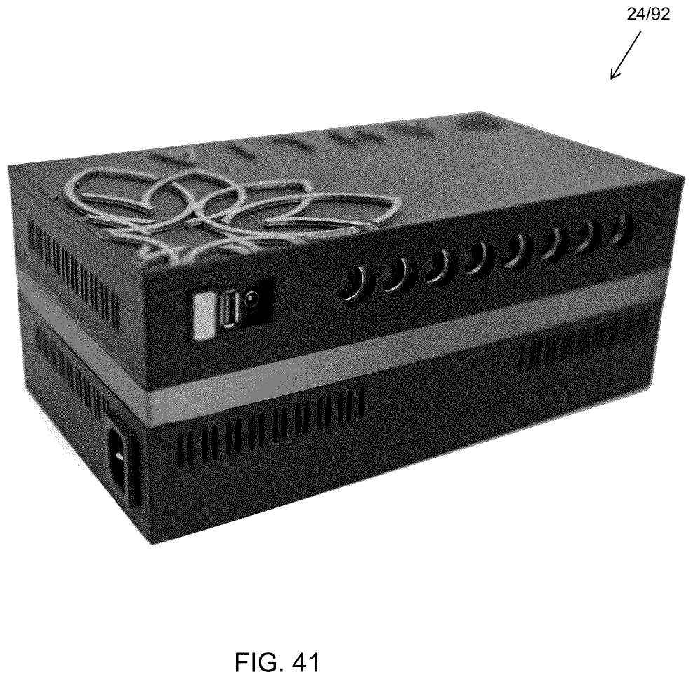

FIG. 42 shows an upper front perspective view of a sanitary cover for a flexible light array, in accordance with one or more arrangements.

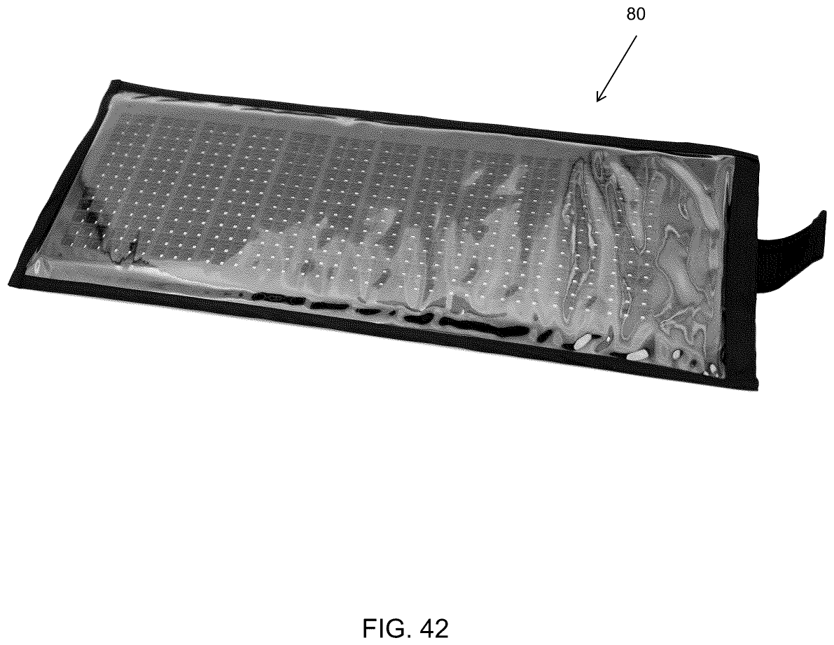

FIG. 43 shows an example prescription for circulation, in accordance with one or more arrangements.

FIG. 44 shows an example prescription for treatment of pain, in accordance with one or more arrangements.

FIG. 45 shows an example prescription for recovery, in accordance with one or more arrangements.

FIG. 46 shows an example prescription for skin care, in accordance with one or more arrangements.

FIG. 47 shows an example prescription for weight loss, in accordance with one or more arrangements.

### DETAILED DESCRIPTION OF THE DISCLOSURE

In the following detailed description of the embodiments, reference is made to the accompanying drawings which form a part hereof, and in which is shown by way of illustration specific embodiments in which the disclosure may be practiced. The embodiments of the present disclosure described below are not intended to be exhaustive or to limit the disclosure to the precise forms in the following detailed description. Rather, the embodiments are chosen and described so that others skilled in the art may appreciate and understand the principles and practices of the present disclosure. It will be understood by those skilled in the art that various changes in form and details may be made without departing from the principles and scope of the invention. It is intended to cover various modifications and similar arrangements and procedures, and the scope of the appended claims therefore should be accorded the broadest interpretation so as to encompass all such modifications and similar arrangements and procedures. For instance, although aspects and features may be illustrated in or described with reference to certain figures or embodiments, it will be appreciated that features from one figure or embodiment may be combined with features of another figure or embodiment even though the combination is not explicitly shown or explicitly described as a combination. In the depicted embodiments, like reference numbers refer to like elements throughout the various drawings.

It should be understood that any advantages and/or improvements discussed herein may not be provided by various disclosed embodiments, or implementations thereof. The contemplated embodiments are not so limited and should not be interpreted as being restricted to embodiments which provide such advantages or improvements. Similarly, it should be understood that various embodiments may not address all or any objects of the disclosure or objects of the invention that may be described herein. The contemplated embodiments are not so limited and should not be interpreted as being restricted to embodiments which address such objects of the disclosure or invention. Furthermore, although some disclosed embodiments may be described relative to specific materials, embodiments are not limited to the specific materials or apparatuses but only to their specific characteristics and capabilities and other materials and apparatuses can be substituted as is well understood by those skilled in the art in view of the present disclosure.

It is to be understood that the terms such as “left, right, top, bottom, front, back, side, height, length, width, upper, lower, interior, exterior, inner, outer, and the like as may be used herein, merely describe points of reference and do not limit the present invention to any particular orientation or configuration.

As used herein, “and/or” includes all combinations of one or more of the associated listed items, such that “A and/or B” includes “A but not B,” “B but not A,” and “A as well as B,” unless it is clearly indicated that only a single item, subgroup of items, or all items are present. The use of “etc.” is defined as “et cetera” and indicates the inclusion of all other elements belonging to the same group of the preceding items, in any “and/or” combination(s).

As used herein, the singular forms “a,” “an,” and “the” are intended to include both the singular and plural forms, unless the language explicitly indicates otherwise. Indefinite articles like “a” and “an” introduce or refer to any modified term, both previously-introduced and not, while definite articles like “the” refer to a same previously-introduced term; as such, it is understood that “a” or “an” modify items that are permitted to be previously-introduced or new, while definite articles modify an item that is the same as immediately previously presented. It will be further understood that the terms “comprises,” “comprising,” “includes,” and/or “including,” when used herein, specify the presence of stated features, characteristics, steps, operations, elements, and/or components, but do not themselves preclude the presence or addition of one or more other features, characteristics, steps, operations, elements, components, and/or groups thereof, unless expressly indicated otherwise. For example, if an embodiment of a system is described as comprising an article, it is understood the system is not limited to a single instance of the article unless expressly indicated otherwise, even if elsewhere another embodiment of the system is described as comprising a plurality of articles.

It will be understood that when an element is referred to as being “connected,” “coupled,” “mated,” “attached,” “fixed,” etc. to another element, it can be directly connected to the other element, and/or intervening elements may be present. In contrast, when an element is referred to as being “directly connected,” “directly coupled,” “directly engaged” etc. to another element, there are no intervening elements present. Other words used to describe the relationship between elements should be interpreted in a like fashion (e.g., “between” versus “directly between,” “adjacent” versus “directly adjacent,” “engaged” versus “directly engaged,” etc.). Similarly, a term such as “operatively”, such as when used as “operatively connected” or “operatively engaged” is to be interpreted as connected or engaged, respectively, in any manner that facilitates operation, which may include being directly connected, indirectly connected, electronically connected, wirelessly connected or connected by any other manner, method or means that facilitates desired operation. Similarly, a term such as “communicatively connected” includes all variations of information exchange and routing between two electronic devices, including intermediary devices, networks, etc., connected wirelessly or not. Similarly, “connected” or other similar language particularly for electronic components is intended to mean connected by any means, either directly or indirectly, wired and/or wirelessly, such that electricity and/or information may be transmitted between the components.

It will be understood that, although the ordinal terms “first,” “second,” etc. may be used herein to describe various elements, these elements should not be limited to any order by these terms unless specifically stated as such. These terms are used only to distinguish one element from another; where there are “second” or higher ordinals, there merely must be a number of elements, without necessarily any difference or other relationship. For example, a first element could be termed a second element, and, similarly, a second element could be termed a first element, without departing from the scope of example embodiments or methods.

Similarly, the structures and operations discussed herein may occur out of the order described and/or noted in the figures. For example, two operations and/or figures shown in succession may in fact be executed concurrently or may sometimes be executed in the reverse order, depending upon the functionality/acts involved. Similarly, individual operations within example methods described below may be executed repetitively, individually or sequentially, to provide looping or other series of operations aside from single operations described below. It should be presumed that any embodiment or method having features and functionality described below, in any workable combination, falls within the scope of example embodiments.

As used herein, various disclosed embodiments may be primarily described in the context of light therapy. However, the embodiments are not so limited. It is appreciated that the embodiments may be adapted for use in other applications which may be improved by the disclosed structures, arrangements and/or methods. The system is merely shown and described as being used in the context of light therapy for ease of description and as one of countless examples.

### System 10

With reference to the figures, a system for therapeutic light treatment 10 (or simply “system 10”) is presented. System 10 is formed of any suitable size, shape, and design and is configured to generate and direct light toward a patient for therapeutic light treatment. In one or more arrangements shown, as one example, system 10 includes a chamber 16, a light arrays (e.g., flexible light array 20 or flexible light array pads 18), and a control system 24, among other various components.

### Light Array Pads 18

Light array pads 18 are formed of any suitable size, shape, and design and are configured to generate light for therapeutic light treatment while providing a comfortable surface to support a patient. In one or more arrangements, as one example, light array pads 18 each include a flexible light array 20 operably connected to a flexible pad 22.

### Flexible Light Array 20

Flexible light array 20 is formed of any suitable size, shape, and design and is configured to generate light for therapeutic light treatment.

### Light Emitters 30:

On one or more arrangements, flexible light array 20 includes a plurality of light emitters 30 positioned in a distributed arrangement across the flexible light array 20. Light emitters 30 are formed of any suitable size, shape, and design and are configured to generate various spectra of therapeutic light. In the arrangement shown, as one example, light emitters 30 are light emitting diodes (LEDs). However, the arrangements are not so limited. Rather, it is contemplated that in some various arrangements, light emitters 30 may utilize various different methods and/or means for generating light including but not limited to, for example, LEDs, incandescent lights, fluorescent lights, and/or any other method or means for generating light).

In one or more arrangements, lights emitters 30, include a set of light emitters configured to emit therapeutic light in the red spectrum. Additionally or alternatively, in one or more arrangements, lights emitters 30, include a set of light emitters configured to emit therapeutic light in the infrared spectrum. In one or more arrangements, the infrared set 34 may include multiple subsets of light emitters 30 configured to emit light in different ranges of the infrared spectrum (e.g., near infrared, shortwave infrared, mid wave infrared, and long wave infrared). However, the arrangements are not so limited. Rather, it is contemplated that in some various arrangements, flexible light array 20 may include light emitters 30 in any number of different additional and/or alternative spectrums of light.

Through careful experimentation and observation, it has been surprisingly discovered that a flexible light array 20 with three sets of light emitters 30 (e.g., 30A, 30B, and 30C), configured to generate therapeutic light at approximately 630 nm, 850, nm, and 940 nm, provides flexibility to generate therapeutic light with various different combinations of wavelength and/or intensity to facilitate treatment for various conditions and/or target outcomes.

In one or more arrangements, different flexible light arrays 20 of system 10 may be configured to emit light of different sets of spectra. For example, in one or more arrangements, a flexible light arrays 20 positioned for treatment of a patient's face may be configured with light emitters that emit set of spectra that is different from those emitted by the other flexible light arrays 20 of the system. As an illustrative example, in one or more arrangements, the flexible light arrays 20 for treatment of a face is configured to emit red light (e.g., 660 nm) so as to avoid long term exposure of eyes to near infra-red or infra-red light.

### Array Segments 40:

In one or more arrangements, light emitters 30 of flexible light array 20 are organized into a plurality of arrays segments 40. Array segments 40 are formed of any suitable size, shape, and design and includes a respective subset of the light emitters 30 distributed thereon.

In an arrangement shown, as one example, each array segment 40 has a respective subset of light emitters 30 arranged in rows 42 and columns 44. In an example arrangement shown, as one example, array segments 40 each have light emitters 30 arranged into twelve rows 42 and three columns 44. However, the arrangements are not so limited. Rather, it is contemplated that in some various arrangements, light emitters 30 may be organized into any number of different rows 42 and columns 44.

In one or more arrangements, light emitters 30 of each array segment 40 have sets of light emitters 30A, 30B, and 30C in a distributed mosaic arrangement with one light emitter 30 of each set 30A, 30B, and 30C in each row 42. In one or more arrangements, the sets of light emitters 30A, 30B, and 30C are shifted one column 44 over in each successive row 42, which positions and/or organizes each set 30A, 30B, and 30C into a series of diagonal rows. This arrangement increases spacing between light emitters of each set 30A, 30B, and 30C and provides an equal number of each set 30A, 30B, and 30C, thereby providing a more even distribution for each emitted wavelength of therapeutic light.

However, the arrangements are not so limited. Rather, it is contemplated that in some arrangements, the various sets of emitters 30 of an array segment 40 may be positioned in various additional or alternative mosaic arrangements. As some non-limiting examples, in some arrangements, light emitters in sets 30A, 30B, and 30C may be arranged in a mosaic similar to, for example, a Bayer mosaic, a Quad Bayer mosaic, an X-trans mosaic, an EXR mosaic, a Kodak RCBW mosaic, a Nonacell mosaic, and/or any other mosaic and/or pattern.

In one or more arrangements, as is shown, array segments 40 of flexible light array 20 are arranged one next to each along a length of flexible light array 20. However, the arrangements are not so limited. Rather, it is contemplated that in some arrangements, array segments 40 may be positioned in various additional or alterative arrangements on flexible light array 20.

In one or more arrangements, as is shown, array segments 40 of flexible light array 20 are formed together on a single flexible circuit board 50 with flexible circuitry 52 interconnecting array segments 40 and/or control circuits (if applicable), such as driver circuits 96, central control circuit 94, and/or other circuits or components of control system 24.

In one or more arrangements, flexible light array 20 optionally includes rigid support members 54 attached to flexible circuit board 50 proximate to each array segment 40. Rigid support members 54 are formed of any suitable size, shape, and design and are configured to provide rigidity and support for array segments 40 during use of flexible light array 20. The rigidity and support provided by rigid support members 54 may help to inhibit bending and/or other wear and tear on circuitry of array segments 40 (e.g., wiring, solder joints, etc.) during use. In one or more arrangements shown, as one example, rigid support members 54 are formed or a rigid nonconductive material and have a generally rectangular planar shape of approximately the same size as array segments 40. However, the arrangements are not so limited. Rather, it is contemplated that various other structures may be utilized to provide rigidity and support for array segments 40.

While some arrangements may be primarily described with reference to a flexible light array 20 having light emitters 30/array segment 40 formed on single flexible circuit board 50, the arrangements are not so limited. Rather, it is contemplated that in some arrangements, light emitters 30/array segment 40 of flexible light array 20 may be formed on two or more separate circuit board segments 58 interconnected by flexible circuitry 52 (e.g., flexible wiring).

### Flexible Pad 22:

In one or more arrangements, flexible light array 20 is encased or embedded in a flexible pad 22. Flexible pad 22 is formed of any suitable size, shape, and design, and is configured to position flexible light array 20 for directing therapeutic light of light emitters 30 toward a patient while providing a comfortable interface between a patient and the flexible light array 20.

In one or more arrangements, as is shown, flexible pad 22 has a generally rectangular planar shape having an upper surface 64 and a lower surface 66 extending between a forward edge 68, a rearward edge 70, and opposing side edges 72. In one or more arrangements, as is shown, flexible light array 20 is encased in flexible pad 22, positioned between upper surface 64 and lower surface 66.

In one or more arrangements, as is shown, flexible pad 22 has a plurality of windows 76 formed in upper surface 64. Windows 76 are formed of any suitable size, shape, and design, and are configured to provide pathways for therapeutic light generated by light emitters 30 to be directed outward from upper surface 64 of flexible pad 22. In the arrangement shown, as one example, windows 76 are openings extending from upper surface 64 of flexible light array 20 to upper surface 64 of flexible pad 22. However, the arrangements are not so limited. For example, it is contemplated that in some alternate arrangements, windows 76 may be formed of transparent materials in addition to or in lieu of openings.

In one or more arrangements, as is shown, windows 76 have an inverted rectangular pyramidal shape with narrow bottoms proximate to light emitters 30 and widening as windows 76 extend upward to rectangular shaped openings in upper surface 64 of flexible light array 20. The pyramidal shape of windows helps to increase the area that therapeutic light is provided to in a treatment area, and thereby providing more even application of therapeutic light, while retaining a comfortable interface between a patient and the flexible light array 20.

### Reflective Coatings/Inserts

In one or more arrangements, flexible pad 22 includes reflective material (e.g., reflective paint, coating, film, or other material) positioned on one or more surfaces within windows 76 to direct therapeutic light out through windows 76. In one or more arrangements, flexible pad 22 includes reflective inserts 78 configured to be positioned within windows 76 and provide a reflective surface to direct therapeutic light out through windows 76. Additionally or alternatively, in one or more arrangements, a unitary reflective cover (not shown) may be placed over flexible pad 22 with inserts configured to fit within windows 76. In one or more example implementations, an exterior facing surface of such unitary reflective cover has a chrome or other reflective coating. In some various arrangements, different flexible pad 22 of system 10 may be configured with different arrangements of reflective materials. For example, in some arrangements, flexible pads 22 positioned on canopy 202 may use a unitary reflective cover while flexible pads 22 positioned on bed 200 may use reflective inserts 78.

### Materials:

In some various different arrangements, flexible pad 22 may be formed of various materials configured to provide comforting support when seated/laid upon by a patient. In some arrangements, such material may include but are not limited to, for example, one or more compressible materials such as rubber, silicon, latex, polyurethane, and/or other flexible materials in various forms including but not limited to solid materials, open cell forms, closed cell foams, textiles, and/or any other form of material.

### Sanitary Cover 80:

In one or more arrangements flexible pad is configured to facilitate placement of a sanitary cover 80 on upper surface 64 of flexible pad 22. Sanitary Cover is formed of any suitable size, shape, and design, and is configured to provide a sanitary surface for contact with a patient while permitting therapeutic light generated by light emitters 30 to pass through sanitary cover 80. In one or more arrangements, as one example, sanitary cover may formed of a transparent plastic, rubber, latex, vinyl, or any other suitable material.

### Control System 24:

Control system 24 is formed of any suitable size, shape, and design and is configured to electronically or communicatively connect with flexible light array 20 to control operation of light emitters 30 in response to user input. In some various different arrangements, control system 24 (and various other functional blocks, modules, controllers, devices, and/or circuits of system 10) may be implemented using various different types of electrical circuits, devices and/or systems (collectively “processing systems”) that are specifically configured to carry out one or more of these or related operations/activities. For example, such processing systems may include discrete logic circuits or programmable logic circuits configured for implementing these operations/activities, as shown in the figures and/or described in the specification. In certain embodiments, such a programmable logic circuit may include one or more programmable integrated circuits (e.g., field programmable gate arrays and/or programmable ICs). Additionally or alternatively, such a programmable logic circuit may include one or more processing circuits/devices (e.g., a computer, microcontroller, system-on-chip, smart phone, tablet, server, and/or cloud computing resources).

### Example Control System 24:

In one or more arrangements shown, as one example, control system 24 includes an on-board control circuit 90 and an external control circuit 92 communicatively connected to the on-board control circuit 90. However, the arrangements are not so limited. Rather, it is contemplated that in some various arrangements on-board control circuit 90 and an external control circuit 92 may be implemented together (e.g., on the same devices and/or circuit board).

### On-board Control Circuit 90

On-board control circuit 90 is formed of any suitable size, shape, and design and is configured to control operation of light emitters 30 in response to control signals received from external control circuit 92 or a user interface. In the arrangement shown, on-board control circuit 90 includes a central control circuit 94 and one or more driver circuits 96.

### Driver Circuits 96:

Driver circuits 96 are formed of any suitable size, shape, and design and are configured to selectably provide power to light emitters 30 of flexible light array 20 to control operation and intensity of light emitters 30. In the arrangement shown, as one example, flexible light array 20 includes a respective driver circuit 96 for each array segment 40. In some various arrangements, driver circuits may be configured to control operation of light emitters 30 by control of array segment 40 as a whole, individual sets of light emitters 30 of an array segment 40 (e.g., 30A, 30B, and 30C), individual rows 42 of an array segment 40, individual columns 44 of an array segment 40, and/or, individual light emitters 30 of an array segment 40. Such control may include but is not limited to, for example, providing power to specific light emitters 30 and/or controlling the amount of power provided to such light emitters 30, among other control functions.

### Central Control Circuit 94:

Central control circuit 94 is formed of any suitable size, shape, and design and is configured to control operation of driver circuits 96 to control operation of light emitters 30 responsive to control signals from external control circuit (or alternative user interface). In one or more arrangements, central control circuit 94 is configured to direct driver circuits 96 to operate light emitters 30 in various wavelength, intensity, waveforms (e.g., solid on, blinking, flashing, pulsating, slow waves, and/or other waveform types), and/or patterns across flexible light array 20 as may be desired. For ease of reference a particular combination of wavelength, intensity, waveforms, and/or patterns for operating light emitters 30 may be referred to as a “prescriptive treatment”.

### External Control Circuit 92:

In one or more arrangements, system 10 optionally includes an external control circuit 92 communicatively connected to on-board control circuit 90 and is configured to provide control signals to on-board control circuit 90 to control operation of light emitters 30 of flexible light array 20 as directed in response to user input via a user interface. In one or more arrangements, external control circuit 92 may additionally provide power to central control circuit 94 and/or driver circuits 96 to facilitate operation of flexible light array 20.

### Sensors 98:

In one or more arrangements, control system 24 is configured to monitor data from one or more sensors 98 to dynamically adjust operation of flexible light array 20 in response to real time operating parameters. For example, in one or more arrangements, system 10 may include sensors 98 configured to monitor temperature of the flexible light, flexible pad, and/or patient. However, the arrangements are not so limited to temperature sensors 98. Rather, it is contemplated that in some various arrangements, system 10 may include various types of sensors relevant to light therapy including by not limited to, for example, temperature sensors, pressure sensors, biometric sensors to measure biometric data of a patient (e.g., skin temperature, body temperature, heart rate, blood pressure, blood oxygen levels, respiratory rate, galvanic skin response, bioelectrical impedance, brain waves, and/or various other biometric parameters).

### Processing System 100:

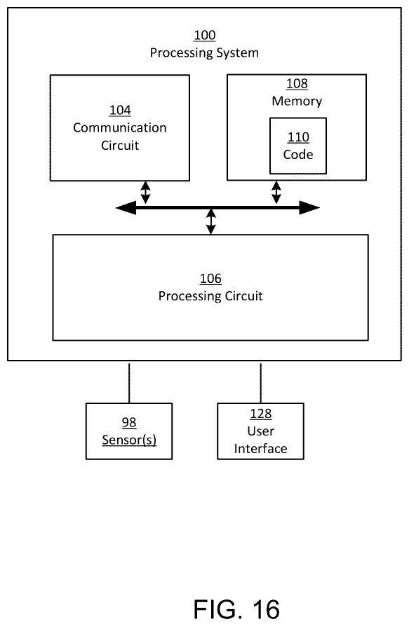

FIG. 16 shows a block level diagram of an example implementation of a processing system 100 that may be utilized for implementation of on-board control circuit 90, external control circuit 92, control system 24 generally, and/or various other components of system 10, consistent with one or more arrangements. In this example, control system 24 is implemented by a processing system 100 having a communication circuit 104, a processing circuit 106, and a memory 108 having software code 110 or instructions that facilitates the operation of system 10, among other components.

Communication circuit 104 is formed of any suitable size, shape, design, technology, and in any arrangement and is configured to facilitate communication with devices to be controlled, monitored, and/or alerted by control system 24. In one or more arrangements, as one example, communication circuit 104 includes a transmitter (for one-way communication) or transceiver (for two-way communication). In various arrangements, communication circuit 104 may be configured to communicate with various components of system 10 (e.g., using various wired and/or wireless communication technologies and protocols over various networks and/or mediums including but not limited to, for example, IsoBUS, Serial Data Interface 12 (SDI-12 ), UART, Serial Peripheral Interface, PCI/PCIe, Serial ATA, MODBUS RTU, ARM Advanced Microcontroller Bus Architecture (AMBA), USB, Firewire, RFID, MODBUS TCP, EtherNet/IP, Near Field Communication (NFC), infrared and optical communication, 802.3/Ethernet, 802.11/ WIFI, Profibus, Wi-Max, Bluetooth, Bluetooth low energy, EtherCAT, Controller Area Network (CAN), Ultra Wideband (UWB), 802.15.4/ZigBee, ZWave, GSM/EDGE, UMTS/HSPA+/HSDPA, CDMA, LTE, RPMA,FM/VHF/UHF networks, and/or any other communication protocol, technology or network.

Processing circuit 106 may be any computing device that receives and processes information and outputs commands according to software code 110 stored in memory 108. For example, in some various arrangements, processing circuit 106 may be discrete logic circuits or programmable logic circuits configured for implementing these operations/activities, as shown in the figures and/or described in the specification. In certain arrangements, such a programmable circuit may include one or more programmable integrated circuits (e.g., field programmable gate arrays and/or programmable ICs). Additionally or alternatively, such a programmable circuit may include one or more processing circuits (e.g., a computer, microcontroller, system-on-chip, smart phone, server, and/or cloud computing resources). For instance, computer processing circuits may be programmed to execute a set (or sets) of software code stored in and accessible from memory 108. Memory 108 may be any form of information storage such as flash memory, RAM memory, DRAM memory, a hard drive, or any other form of memory.

Processing circuit 106 and memory 108 may be formed of a single combined unit. Alternatively, processing circuit 106 and memory 108 may be formed of separate but electrically connected components. Alternatively, processing circuit 106 and memory 108 may each be formed of multiple separate but communicatively connected components.

Software code 110 is any form of instructions or rules that direct processing circuit 106 how to receive, interpret and respond to information to operate as described herein. Software code 110 or instructions are stored in memory 108 and accessible to processing circuit 106.

### User Interface 128:

User interface 128 is formed of any suitable size, shape, design, technology, and in any arrangement and is configured to interact with control system 24 to facilitate input, access to, and processing of relevant data related to operation of system 10. In one or more arrangements, as one example, user interface 128 includes a set of inputs 130. Inputs are formed of any suitable size, shape, design, and technology and are configured to facilitate user input of data and/or control commands. In various different arrangements, inputs may include various types of controls including but not limited to, for example, buttons, switches, dials, knobs, a keyboard, a mouse, a touch pad, a touchscreen, a joystick, a roller ball, microphone (e.g., voice recognition), or any other form of user input. Optionally, in one or more arrangements, user interface 128 includes a display 132. In one or more arrangements shown, as one example, user interface 128 utilizes a touchscreen display, which operates as both inputs 130 and display 132.

In some various arrangements, user interface 128 may be provided by on-board control. circuit 90, by external control circuit 92, or by another portion of control system 24. Additionally or alternatively, in some arrangements, user interface may be provided by a user device (e.g., smartphone, tablet, laptop, computer, or other user device) that is communicatively connected to control system 24.

### Chamber 16:

In one or more arrangements, as is shown, system 10 includes a chamber 16 to facilitate therapeutic light treatment of a patient. Chamber 16 is formed of any suitable size, shape, and design and is configured to position a plurality of light array pads 18 and/or flexible light arrays 20 for therapeutic light treatment of a patient located within the chamber 16. As some non-limiting examples, chamber 16 may be configured with a bed for treatment of a patient in a lying position, as a booth for treatment of a patient in a seated or standing position, or any other configuration for treatment of patients in any other suitable position.

In one or more arrangements, as is shown, system 10 includes a chamber 16 having a bed 200, a canopy 202, a frame 204, and a plurality of plurality of light array pads 18 and/or flexible light arrays 20 operably connected to the bed 200, canopy 202, and/or other component of chamber 16.

### Bed 200:

Bed 200 is formed of any suitable size, shape, and design and is configured to provide a surface for support of a patient and providing one or more light array pads 18 thereon. In the arrangement shown, bed 200 includes a plurality of support sections 208 operably connected to frame 204. In this example arrangement, support sections 208 have an elongated rectangular generally planar shape having an upper surface 212 extending between a forward edge 214, a reward edge 216, and opposing side edges 218.

However, the arrangements are not so limited. Rather, it is contemplated that in some various arrangements, support sections 208, may have various alternative shapes suitable for support of a patient and/or light array pads 18. Moreover, it is contemplated that in some arrangements, various support sections 208 (or all support sections) may be formed as unitary components providing a continuous upper surface 212.

In the arrangement shown, support sections 208 are positioned by frame 204 to support a patient in a reclined sitting position with legs elevated. For example, in this example arrangement, support sections 208 are positioned in a row to form a larger generally rectangular shaped bed 200, when viewed from the top, having a segmented upper surface 222 extending between opposing side edges 224 from a head end 226 to a foot end 228. In this example arrangement, upper surface 222 of bed 200 has an angled back support section 230, an angled seat support section 232, and a horizontal leg support section 234.

However, the arrangements are not so limited. Rather, it is contemplated that in some various different arrangements, support sections 230/232/234 and/or bed 200 may be configured to support a patient in any additional or alternative position including but not limited to for example: a reclined sitting position, a Fowler's position, a semi-Fowler's position, a prone position, a supine position, or any other position.

### Canopy 202:

Canopy 202 is formed of any suitable size, shape, and design and is configured to facilitate positioning of plurality of light array pads 18 and/or flexible light arrays 20 above a patient. In the arrangement shown, as one example, canopy 202 has a generally rectangular planar shape having a top 240, bottom 242 and opposing sides 244 extending between a front 246 and a back 248. In the arrangement shown, as one example, canopy 202 has sloped and horizontal sections that are respectively generally parallel to back support section 230, seat support section 232, and leg support section 234 of bed 200 when canopy 202 is lowered to a closed position. In this arrangement, light array pads 18 and/or flexible light arrays 20 connected to canopy 202 are positioned for light therapy of a patient supported by bed 200 in a reclined sitting position with legs elevated. However, the arrangements are not so limited. Rather, it is contemplated that in some various different arrangements, canopy 202 may be configured to position light array pads 18 and/or flexible light arrays 20 for light therapy of a patient supported by bed 200 in any additional or alternative position including but not limited to for example: a reclined sitting position, a Fowler's position, a semi-Fowler's position, a prone position, a supine position, or any other position. In one or more arrangements, canopy 202 may be adjustable in shape so as to better conform to different body types. For example, in one or more arrangements, canopy 202 may be formed of a plurality of sections that are connected together by hinged connectors that allow relative angle of the sections to be adjusted.

### Frame 204

Frame 204 is formed of any suitable size, shape, and design and is configured to operably interconnect and support bed 200, canopy 202, and/or other components of system 10. In this example arrangement, as one example, frame includes a front support stand 260, a rear support stand 262, a bed support member 264 extending between the front support stand 260 and the rear support stand 262, and a canopy support member 266 pivotally connected to the rear support stand 262.

### Rear Support Stand 262:

Rear support stand 262 is formed of any suitable size, shape, and design and is configured to operably connect with and support canopy support member 266 on the floor. In one or more arrangements, as is shown, rear support stand 262 has a generally rectangular shaped structure with a hollow interior formed by a front wall 272, a rear wall 274, a top 276, a bottom 278, and open sides 280. In this example arrangement, rear support stand 262 includes one or more shelves 282 extending between front wall 272 and rear wall 274 within the hollow interior 270.

In this example arrangement, front wall 272, rear wall 274, top 276, bottom 278, shelves 282 and/or other components of rear support stand 262 are interconnected and reenforced by a plurality of rigid support members. In the arrangement shown, as one example, rigid support members are formed of rectangular piping. However, the arrangements are not so limited. Rather, it is contemplated that various components of rear support stand 262 may be interconnected and supported by various methods and/or means including but not limited to various shaped pipes, rods, bar stock, beams and/or other elongated members, which may be formed of various suitable materials including but not limited to metals, plastics or other polymers, wood and/or other organic materials, composites and/or any other suitable material.

### Front Support Stand 260:

Front support stand 260 is formed of any suitable size, shape, and design and is configured to operably connect with bed support member 264 for support of bed 200 on the floor. In this example arrangement, as one example, front support stand 260 includes a generally planar oval shaped base 292 configured to facilitate contact with the floor with a collar 296 on an upper surface 294 of base 292 to facilitate connection with lower end xxx of bed support member 264.

In this example, collar 296 has a set screw 298 connector to secure the connection with bed support member 264. However, the arrangements are not so limited. Rather, it is contemplated that in some various arrangements, base may be implemented using various shaped structures and connect with vertical support using various methods and/or means to facilitate connection including but not limited to, for example, screws, bolts, clamps, pins, rivets, welding, snap connectors, friction fit connectors, adhesives, chemical bonding, and/or any other process or means that results in a permanent of semi-permanent connection.

### Bed Support Member 264:

Bed support member 264 is formed of any suitable size, shape, and design and is configured to extend between the front support stand 260 and the rear support stand 262 and support bed 200 thereon. In the arrangement shown, as one example, bed support member 264 is formed by an elongated cylindrical pipe extending forward from a rearward end 310, where bed support member 264 connects to rear support stand 262, under bed 200, and then extending vertically downward to a lower end 312, where bed support member 264 connects with front support stand 260.

In this example arrangement, bed support member 264 has an angled back support section 314, an angled seat support section 316, and a horizontal leg support section 318 positioned in general alignment with angled back support section 230, an angled seat support section 232, and a horizontal leg support section 234 of bed 200. In this example arrangement, bed support member 264 has a vertical support section extending vertically upward from lower end 312 to a forward end 320 of angled back support section 230. In the arrangement shown, bed support member 264 is formed of a continuous unitary piece of pipe. However, the arrangements are not so limited. Rather, it is contemplated that in some various arrangements, bed support member 264 may be formed from a plurality of separate segments that are joined together.

### Canopy Support Member 266:

Canopy support member 266 is formed of any suitable size, shape, and design and is configured to pivotally connect canopy 202 with rear support stand 262 and facilitate movement of canopy 202 between a raised position and a lowered position.

In the arrangement shown, as one example, canopy support member 266 is formed by an elongated cylindrical pipe extending between a rearward end 332, where canopy support member 266 is pivotally connected to rear support stand 262, to a forward outward end 336 where canopy 202 is held in a cantilevered position.

In this example arrangement, canopy support member 266 extends rearward from rearward end 332, curves and extends upward to an elevated position, and then curves and extends forward to the canopy 202. In this example arrangement, canopy support member 266 extends through canopy from back 248 to front 246, along a center line following the general shape of the horizontal and sloped portions of canopy 202 to facilitate connection with and support of canopy 202.

In one or more arrangements, canopy 202 is connected to canopy support member 266 by one or more fasteners (e.g., bolts). However, the arrangements are not so limited. Rather, it is contemplated that in some various arrangements canopy 202 may be connected with canopy support member 266 using various methods and/or means to facilitate connection including but not limited to, for example, screws, bolts, clamps, pins, rivets, welding, snap connectors, friction fit connectors, adhesives, chemical bonding, and/or any other process or means that results in a permanent of semi-permanent connection.

### Bias Member 340:

In one or more arrangements, system 10 includes a bias member 340. Bias member 340 is formed of any suitable size, shape, and design and is configured to attach to and between canopy support member 266 and rear support stand 262 and provide a bias force to canopy support member 266 (or other component connected thereto). In one or more arrangements, bias member 340 is configured to provide a bias force sufficient to counterbalance the weight of canopy 202 and hold canopy 202 in place in a raised position, lowered position, or any position in between in absence of an opposing force.

In the arrangement shown, as one example, bias member 340 is a telescoping unit having a forward end 342 and a rearward end 228 that have holes there through that serve as a pivot point when raising or lowering canopy 202. In this example arrangement, bias member 340 is configured to extend forward end 342 and rearward end 228 away from each other in absence of an opposing force. In the arrangement shown, as one example, forward end 342 of bias member 340 is connected to pivot connector 346 connected to rear support stand 262 and rearward end 344 of bias member 340 is connected to pivot connector 348 connected to canopy support member 266.

In various arrangements, bias member 340 may utilize various mechanisms to extend forward end 342 and rearward end 344 outward from each other including but not limited to, for example one or more springs, one or more gas pistons, one or more gas springs, one or more hydraulic pistons, one or more actuators, one or more solenoids, one or more pneumatic members, and/or any other force generating means or combination thereof.

In one or more arrangements, the length and/or pressure or strength of bias member 340 is adjustable. This may be accomplished, for example, by rotating one portion of the bias member 340 (such as the lower end or upper end) relative to another portion of the bias member 340 (such as the lower end or upper end). However, the arrangements are not so limited. Rather, it is contemplated that in some various arrangements, various different methods and/or means may be utilized to facilitate adjustment of force applied by bias member 340.

### Contoured Light Array Pads 18.

In one or more arrangements, light array pads 18 positioned on canopy 202 and/or bet 200 may be contoured based on the shape of a human body to position light emitters 30 at a more consistent distance from the body of a patient and thereby provide more uniform application of light. For example, in one or more arrangements, as is shown, the bottom 242 of canopy 202 is curved to orient light emitters 30 closer toward the sides of the patient.

### Control Processes 400:

As an illustrative example, in one or more arrangements, software code 110 or instructions may configure processing circuit 106 to perform various processes (e.g., processes 400) to facilitate control and/or operation of flexible light array 20 and/or provide other features and/or functions performed by system 10.

As some illustrative examples, some processes 400 that may be performed and/or initiated by control system 24 in response to user input from user interface 128 and/or data from sensors 98 include but are not limited to, for example, operation of flexible light array 20, patient monitoring, user and/or prescription authorization, processes for a user interface 128, and/or any other process or action.

While one or more arrangements may primarily describe processes 400 as being performed by control system 24, the arrangements are not so limited. Rather, it is contemplated that in some arrangements, various such processes may be performed whole or in part by one or more systems communicatively connected to control system 24 (e.g., a user smartphone or other device, using cloud-based computing resources, and/or other third-party systems).

### Operation of Light Array Pads:

In one or more arrangements, system 10 may include multiple light array pads 18 and/or flexible light arrays 20 (e.g., in chamber 16) that are operated together to provide therapeutic light therapy across a larger area. In some arrangements, such light array pads 18 and/or flexible light arrays 20 may be controlled independently of each other. In some other arrangements, such light array pads 18 and/or flexible light arrays 20 may be controlled in coordination with one another (e.g., by a shared or master control system 24). However, the arrangements are not so limited. Rather, it is contemplated that in some various arrangements a light array pad 18 and/or flexible light array 20 may be operated individually to provide light therapy treatment over a smaller targeted area.

### Prescriptive Treatments:

In one or more arrangements, control system 24 is configurable via user interface 128 to operate flexible light array 20 or one or more light array pads 18 to generate therapeutic light for various different prescriptive treatments that may be selected by the user and/or clinician. As previously described, a prescriptive treatment directs control system 24 to operate light emitters 30 of flexible light array 20 with a particular set of wavelength, intensity, duration, waveforms (e.g., solid on, blinking, flashing, pulsating, slow waves, and/or other waveform types), and/or patterns across flexible light array 20. Different prescriptive treatments are used to perform light therapy to treat various different conditions (e.g., diabetes, poor circulation, weight loss, skin conditions, etc.).

In some instances, prescriptive treatments may be a static treatment having a consistent waveform pattern throughout the treatment. In some other instances, prescriptive treatments may be sequential treatments having a sequence of different waveform patterns throughout the treatment. Additionally or alternatively, in some other instances, prescriptive treatments may be adaptive treatments that dynamically adapted waveform patterns that change in real time (e.g., based on feedback data from sensors 98).

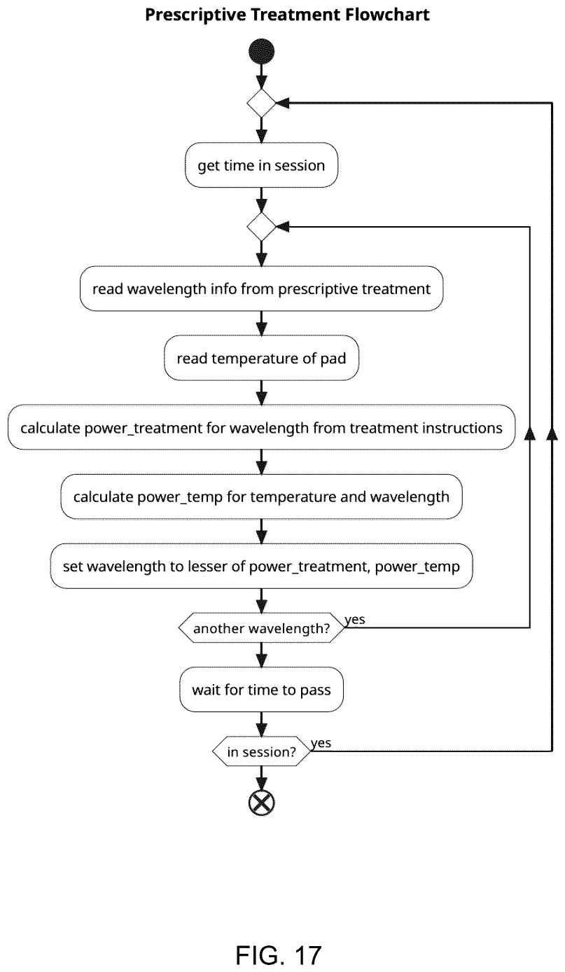

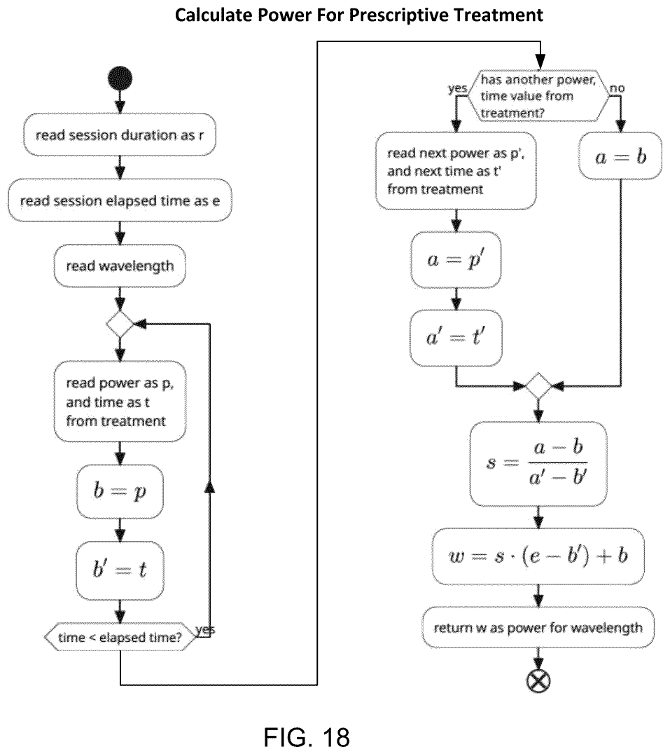

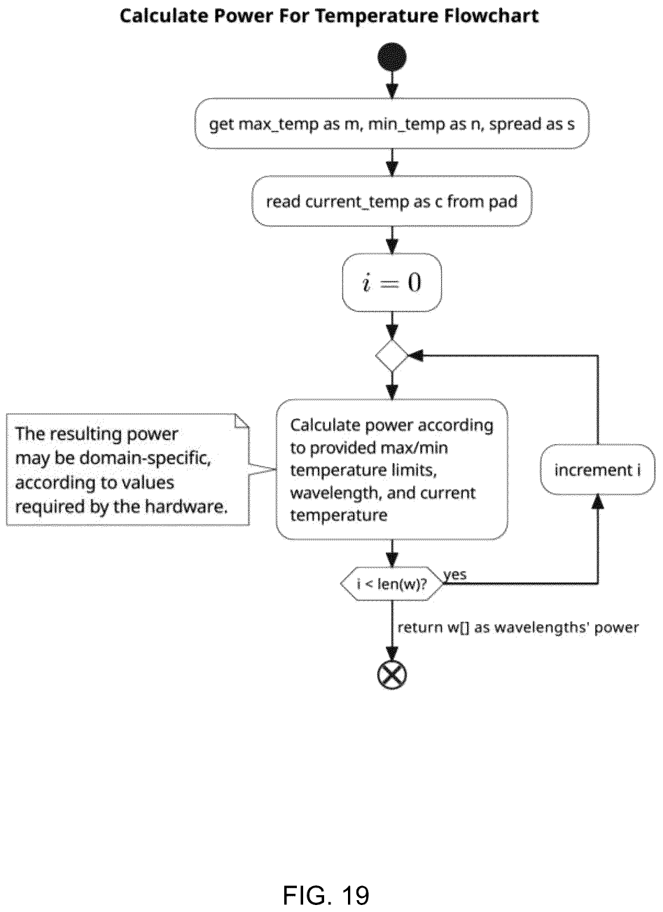

As an illustrative example, FIG. 17 shows a flowchart of an example process for controlling operation of light emitters 30 of flexible light array 20 to generate therapeutic light for a selected prescription treatment, in accordance with one or more arrangements. FIG. 18 shows a flowchart of an example process for determining an amount of power for a particular prescription treatment in performance of the example process shown in FIG. 17, in accordance with one or more arrangements. FIG. 18 shows a flowchart of an example subprocess for determining an amount of power for a particular prescription treatment in performance of the process shown in FIG. 17, in accordance with one or more arrangements. FIG. 19 shows a flowchart of an example subprocess for adjusting power for light emitters 30 based on temperature readings of sensors 98 in performance of the process shown in FIG. 17, in accordance with one or more arrangements.

However, the arrangements are not limited to these example processes. Rather, it is contemplated that in some various arrangements, control system 24 may be configured to generate therapeutic light for a selected prescription treatment using various additional or alternative processes.

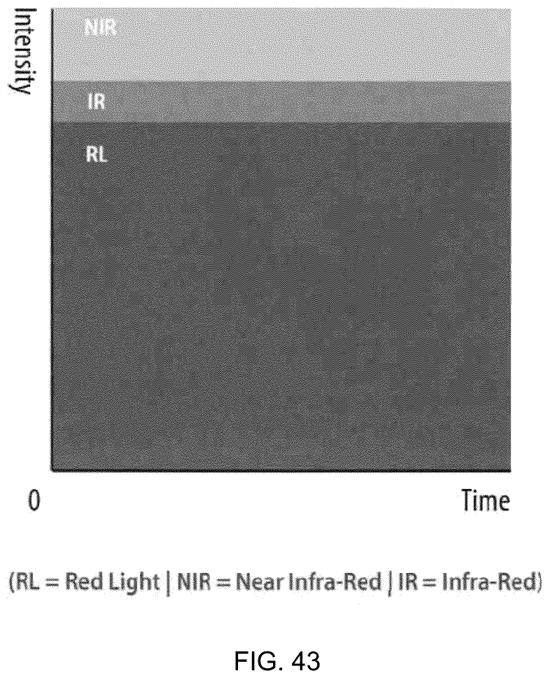

As some illustrative examples, FIGS. 43-47 show intensities of red light (RL), near infra-red light (NIR), and infra-red light (IR) over time for some example prescriptions for various applications.

FIG. 43 shows an example prescription for circulation, which focuses on improving blood flow and vascular function. Dahlia's IR and NIR are elevated in the session to enhance overall circulatory health and amplify results.

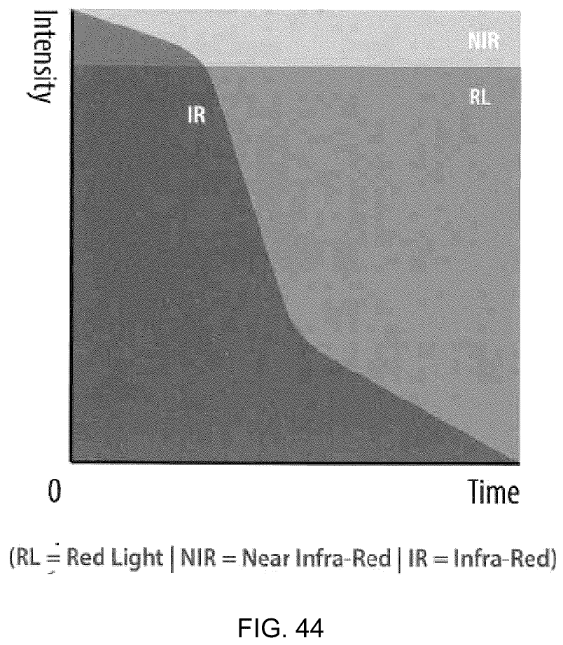

FIG. 44 shows an example prescription for treatment of pain. NIR and red light are the primary application in this therapy. IR starts high to help boost circulation and tapers down to let the NIR continue its support at the cellular level. These wavelengths work together focusing on pain reduction and swelling management.

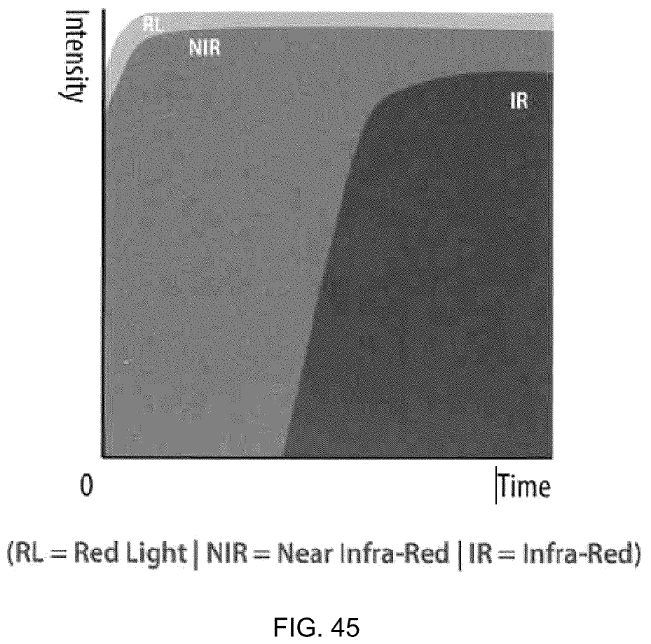

FIG. 45 shows an example prescription for recovery. In this example, RL and NIR are applied at the start of the session. Later in the section, IR is elevated later in the session to increase blood flow in order to amplify results.

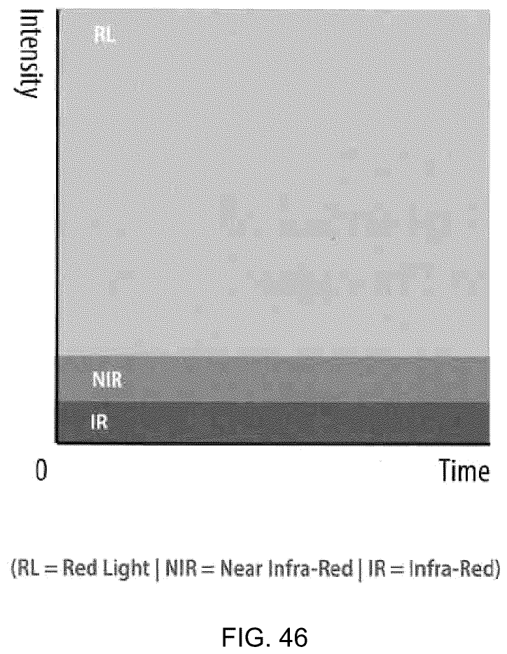

FIG. 46 shows an example prescription for skin care. In this example, RL is the primary application, focusing on collagen stimulation and skin rejuvenation. NIR is added to help tissue oxygenation and microcirculation.

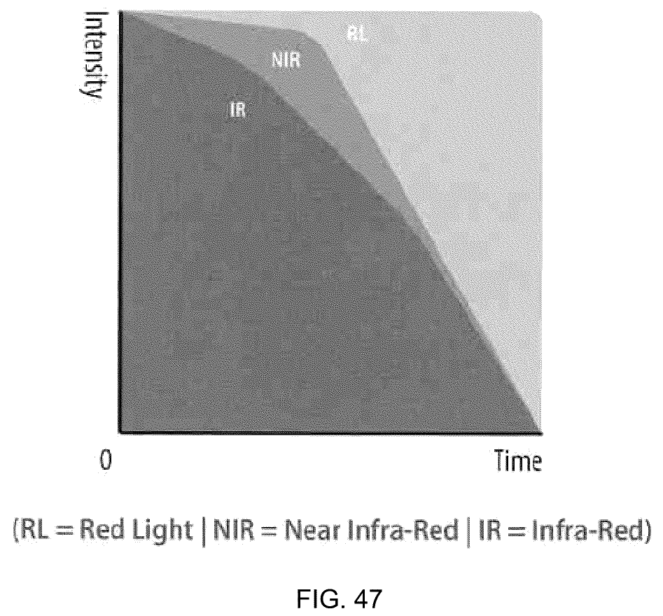

FIG. 47 shows an example prescription for weight loss. In this example, RL, NIR and IR are all applied at the start of the session, focusing on warming up the triglyceride to help the red light lyse the outer shell aiding in fat cell reduction and metabolism enhancement. This allows the contents to liquify and seep into the interstitial fluid where they will be packaged up for excrement. NIR and IR reduced are then reduced over the session.

### Temperature Sensor and Control:

In one or more arrangements, system 10 includes one or more temperature sensors 98. It is recognized that during operation a patient may be in close contact with flexible pad 22 in some areas and may be separated from flexible pad 22 in other areas. For example, a patient laying on flexible pad 22 may be in close contact with flexible pad 22 in an upper back area while the lower back of the patient is lifted a distance off of the flexible pad 22. In such a position of patient, more heat may build up on the upper back area due to lack of air circulation in comparison to the lower back area.

In one or more arrangements, control processes 400 performed by control system 24 may monitor temperature sensor data in different areas of a flexible light array pad 20/22 (and/or in different light array pads 18) and dynamically adjust intensity of light emitters 30 in the different areas (and/or light array pads 18), for example, to prevent overheating and/or maintain a more uniform temperature across the treatment area of the patient. In one or more arrangements, control processes 400 dynamically adjust intensity of light emitters 30 in the different areas (and/or light array pads 18) to maximize light intensity while preventing temperature from exceeding a specified temperature threshold so as to prevent burning and/or discomfort of the patient. In one or more arrangements, the temperature threshold may be adjusted by a patient so as to make a session more comfortable. In one or more arrangements, upon a patient adjusting a temperature threshold, control processes 400 dynamically adjust a duration of the session so as to provide a target cumulative amount of light to the patient.

### Multiple Stage Prescriptions:

In one or more arrangements, system 10 may be operated according to prescriptive treatments having multiple stages. As an illustrative example, a multiple stage prescription may use a first spectrum and/or intensity for a first duration of time to target treatment of surface layers of skin and use a second spectrum and/or intensity for a second duration of time to target treatment of deeper layers of skin. However, the arrangements are not so limited to two prescription stages. Rather, it is contemplated that in some various arrangements, system 10 may be operated according to prescriptions having any number of different stages.

In some arrangements, system 10 may be configured to operate all light array pads 18 and/or flexible light arrays 20 in the same prescription stages at the same time. However, the arrangements are not so limited. Rather, it is contemplated that in some arrangements, system 10 may be configured to rotate light array pads 18 and/or flexible light arrays 20 through prescription stages. As an illustrative example, a hypothetical prescription may be configured to alternate for 30 minutes between a stage 1 and a stage 2 with one min duration in each stage before alternating. Light array pads 18 and/or flexible light arrays 20 could be rotated, for example, between even positioned light array pads 18 and/or flexible light arrays 20 and odd positioned light array pads 18 and/or flexible light arrays 20. In this hypothetical example, when even positioned light array pads 18 and/or flexible light arrays 20 are operated in stage 1, odd positioned light array pads 18 and/or flexible light arrays 20 are operated in stage 2. Conversely, when even positioned light array pads 18 and/or flexible light arrays 20 are operated in stage 2, odd positioned light array pads 18 and/or flexible light arrays 20 are operated in stage 1.

Rotation of light array pads 18 and/or flexible light arrays 20 through stages may help prevent a patient from feeling overheated if a particular stage is significantly warmer than another. By simultaneously operating some light array pads 18 and/or flexible light arrays 20 in the warmer stage and some light array pads 18 and/or flexible light arrays 20 in the cooler stage, the cumulative heat provided to the patient at a given time is reduced. Such rotation may additionally or alternatively be used to provide the patient sensation of moving waves of heat/light or other sensation caused by the transition between prescription stages.

### Dynamic Adjustment of Prescription:

In one or more arrangements, system 10 is configured to adjust (or permit patients to adjust) certain settings and/or modes during treatment. For example, in some various arrangements, system 10 may be configured to permit a patient to adjust wavelength, intensity, waveforms, patterns, stage rotation modes/patterns, and/or various other parameters. In some arrangements, prescriptions may be configured to lock certain parameters (e.g., wavelength) while permitting a patient to adjust others (e.g., intensity). As another example, in some arrangements, control processes 400 may automatically adjust power based on temperature measurements (e.g., to ensure temperature does not exceed a maximum temperature threshold and/or fall below a minimum temperature threshold as shown in FIG. 19).

In one or more arrangements, control processes 400 are configured to dynamically adjust duration of treatment stages and/or cumulative treatment length to ensure a patient is exposed to a particular wavelength for a prescribed cumulative intensity. As an illustrative example, a simple prescription may cause system 10 to expose the patient to a single wavelength of light at a power corresponding to a light intensity of 100 mW/cm2 for 20 minutes (i.e., 2000mWMinutes/cm2). After 10 minutes, the patient may feel too hot and prompt system 10 to adjust power to reduce light intensity by half to 50 mW/cm2. In response to the adjustment by user and/or control processes 400, control processes 400 may be configured to automatically adjust the treatment time so as to cause the patient to receive the prescribed cumulative intensity of the prescribed wavelength. In the above hypothetical example, control processes 400 would increase duration by 10 minutes so as to cause the patient to receive 2000 mWMinutes/cm2 of the prescribed wavelength.

### Localized Prescriptions:

In one or more arrangements, system 10 may simultaneously operate different light array pads 18 and/or flexible light arrays 20 (or portions thereof) according to different prescriptive treatments. As an illustrative example, in one or more arrangements, system 10 may operate a flexible light array pad 20/26 located in canopy 202 above a patient's head according to a first prescription for treatment of acne while operating other light array pads 18 and/or flexible light arrays 20 according to second prescription to promote weight loss. However, the arrangements are not so limited to two treatment zones. Rather, it is contemplated that in some various arrangements, system 10 may be operated with any number of different treatment zones and/or prescriptions.

### Safety Overrides:

In one or more arrangements, control processes 400 are configured to override control signals provided by user interface 128 or external control circuit 92 to prevent risk of harm or discomfort to a patient or damage to system hardware. As an illustrative example, in one or more arrangements, control processes 400 may enforce a rule to setting a maximum permitted intensity of light emitters 30 in certain wavelengths. As another example, in one or more arrangements, control processes 400 may enforce a rule to set a maximum operating temperature threshold by limiting/reducing intensity of light emitters 30 if a temperature indicated by a temperature sensor 98 exceeds the threshold. However, the arrangements are not limited to these illustrative examples. Rather, it is contemplated that in some various arrangements, control processes 400 may be configured to enforce safety override controls based on any criteria or trigger conditions.

In one or more arrangements, safety overrides may be hard coded in control processes 400. Additionally or alternatively, in some arrangements, safety overrides performed by control processes 400 may be customized by an authorized user, for example, via user interface 128. Control processes 400 may perform safety override in various ways. In one or more arrangements, control processes 400 may perform safety overrides on an individual flexible light array pad 20/26 basis. Additionally or alternatively, in one or more arrangements, control processes 400 may perform safety overrides jointly for all flexible light array pad 20/26. For instance, in some arrangements, sensor data from one flexible light array pad may prompt control processes 400 to adjust operation of multiple light array pads 18 and/or flexible light arrays 20. Furthermore, it is contemplated that such adjustment may be specifically tailored to each of multiple light array pads 18 and/or flexible light arrays 20 based on the current operating conditions and sensor data or those individual light array pads 18 and/or flexible light arrays 20.

### User Authorization:

In one or more arrangements, control system 24 is configured to utilize authenticated communication for controlling operation of the system 10. For example, in one or more arrangements, on-board control circuit 90 is configured to authenticate control commands (e.g., received from external control circuit and/or user interface 128) before performing the indicated operation. Such authentication may help reduce the risk of unintended/malicious user, for example, due to signal interference or attempted hacking.

Additionally or alternatively, in one or more arrangements, user interface 128 is configured to authenticate users before the users are permitted to control operation of system 10. Such authentication of users may be useful for doctors to restrict use of system 10 to an intended set of prescriptions for a particular patient and/or prevent use that may be potentially dangerous to a patient.

In some various arrangements, control system 24 may utilize various means and/or methods for authentication of control signals and/or users. As one example, in one or more arrangements, control system 24 is configured to use a token-based authentication to verify that control signals received by on-board control circuit 90 are valid. For example, in one or more arrangements, on-board control circuit 90 may require external control circuit and/or user interface 128 periodically communicate a token to permit or continue operation of the flexible light array pad 20/26. Additionally or alternatively, in some arrangements, on-board control circuit 90 may authenticate individual control commands using a token communicated with the control command.

However, the arrangements are not limited to token-based authentication. Rather, it is contemplated that in some various arrangements, control system 24 may utilize any suitable method or means for authentication including but not limited to, for example, authentication tokens, password authentication protocols, symmetric key authentication, biometric authentication, and/or any other method or means for authentication.

### Objectives Met:

From the above discussion it will be appreciated that system 10 presented herein improves upon the state of the art. Specifically, in one or more arrangements, a an improved system and method for therapeutic light treatment is provided: that applies therapeutic light evenly to a target area; that can apply a plurality of types of therapeutic light; that can apply different prescriptions of therapeutic light patterns for various different treatments; that reduces patient risk; that is sanitary; that is effective; that is reliable; that has a long useful life; and/or that is easy to use. These and other objects, features, or advantages of the disclosure will become apparent from the specification, figures, and claims.

It will be appreciated by those skilled in the art that other various modifications could be made to the device without parting from the spirit and scope of this disclosure. All such modifications and changes fall within the scope of the claims and are intended to be covered thereby.

What is claimed:

1. A system for therapeutic light treatment, comprising:
a chamber;
the chamber configured to house a patient therein;
a plurality of flexible light array pads;
the plurality of flexible light array pads positioned in the chamber so as to expose the patient to therapeutic light when operated;
a control system;
the control system configured to control operation of the plurality of flexible light array pads so as to expose the patient to therapeutic light according to a prescription selected by a user.

2. The system of claim 1, wherein the prescription specifies wavelength, intensity, and duration of the light to be emitted by the plurality of flexible light array pads.

3. The system of claim 1, wherein the prescription specifies wavelength, intensity, duration, and waveforms of the light to be emitted by the plurality of flexible light array pads.

4. The system of claim 1, wherein the prescription specifies wavelength, intensity, duration, and patterns of the light to be emitted by the plurality of flexible light array pads.

5. The system of claim 1, wherein the prescription specifies a plurality of stages of light therapy treatment;
the plurality of stages specifying respective sets of: wavelength, intensity, duration, waveforms and/or patterns of the light to be emitted by the plurality of flexible light array pads in the plurality of stages.

6. The system of claim 1, wherein system is configured to:
determine power and duration for one or more wavelengths specified in the prescription;
determine a current temperature of the plurality of flexible light array pads;
determine an adjusted power based on the determined power and the current temperature of the plurality of flexible light array pads;
operate the plurality of flexible light array pads at the adjusted power.

7. The system of claim 1, wherein the chamber includes a bed and a canopy;
wherein a first set of the plurality of flexible light array pads are positioned on an upper surface of the bed;
wherein a second set of the plurality of flexible light array pads are positioned on a lower surface of the canopy.

8. The system of claim 1, wherein the chamber includes a bed and a canopy;
wherein a first set of the plurality of flexible light array pads are positioned on an upper surface of the bed;
wherein a second set of the plurality of flexible light array pads are positioned on a lower surface of the canopy;
wherein the bed includes a angled back support section, an angled, seat support section, and an approximately horizontal leg support section.

9. The system of claim 1, wherein the chamber includes a bed and a canopy;
wherein the canopy is operably connected to a cantilever arm extending over the bed.

10. The system of claim 1, wherein the chamber includes a bed and a canopy;
wherein the canopy is operably connected to a cantilever arm extending over the bed;
wherein the cantilever arm is configured to pivot relative to the bed to move the canopy between a raised position and a lowered position.

11. The system of claim 1, wherein the chamber includes a bed and a canopy;
wherein a first set of the plurality of flexible light array pads are positioned on an upper surface of the bed;
wherein a second set of the plurality of flexible light array pads are positioned on a lower surface of the canopy;
wherein the canopy is configured to moved between a raised position and a lowered position.

12. The system of claim 1, wherein the plurality of flexible light array pads include a first set of light emitters configured to emit light in a first spectrum and a second set of light emitters configured to emit light in a second spectrum;
wherein the second spectrum is different from the first spectrum.

13. The system of claim 1, wherein the plurality of flexible light array pads include a first set of light emitters configured to emit light in a first spectrum, a second set of light emitters configured to emit light in a second spectrum, and a third set of light emitters configured to emit light in a third spectrum;
wherein the first spectrum, the second spectrum and the third spectrum are mutually exclusive.

14. The system of claim 1, wherein the plurality of flexible light array pads include a first set of light emitters configured to emit light in a red spectrum, a second set of light emitters configured to emit light in a near infrared spectrum, and a third set of light emitters configured to emit light in an infrared spectrum.

15. The system of claim 1, wherein the plurality of flexible light array pads include a first set of light emitters configured to emit light in a 630 nm spectrum, a second set of light emitters configured to emit light in a 940 nm spectrum, and a third set of light emitters configured to emit light in a 950 nm spectrum.

16. The system of claim 1, wherein the flexible light array pads have respective sets of windows formed in a patient facing surface;
wherein the plurality of flexible light array pads have respective sets of light emitters positioned in the respective sets of windows.

17. The system of claim 1, wherein the flexible light array pads have respective sets of windows formed in a patient facing surface;
wherein the plurality of flexible light array pads have respective sets of light emitters positioned in the respective sets of windows;
wherein the plurality of windows are recessed openings having an inverted pyramidal shape.

18. The system of claim 1, wherein the plurality of flexible light array pads have respective sets of windows formed in a patient facing surface;
wherein the plurality of flexible light array pads have respective sets of light emitters positioned in the respective sets of windows;
wherein the sets of windows are recessed openings have a cone shape.

19. The system of claim 1, wherein the plurality of flexible light array pads have respective sets of windows formed in a patient facing surface;
wherein the plurality of flexible light array pads have respective sets of light emitters positioned in the respective sets of windows;
plurality of flexible light array pads have reflective inserts positioned in the plurality of flexible light array pads.

20. A system for therapeutic light treatment, comprising:
a chamber;
the chamber configured to house a patient therein;
a plurality of flexible light array pads;
the plurality of flexible light array pads positioned in the chamber so as to expose the patient to therapeutic light when operated;
wherein the plurality of flexible light array pads are configured to emit red light, near infra-red light, and intra-red light.

21. The system of claim 20, further comprising a control system;
wherein the control system is configured to control operation of the plurality of flexible light array pads so as to expose the patient to therapeutic light according to a prescription selected by a user.

22. The system of claim 20, further comprising a control system;
wherein the control system is configured to determine a respective temperature of each of the plurality of light array pads;
wherein the control system is configured to dynamically adjust operation of the plurality of light array pads based on the determined respective temperatures.

23. The system of claim 20, further comprising a control system;
wherein the control system is configured to determine a respective temperature of each of the plurality of light array pads;
wherein the control system is configured to dynamically adjust operation of the plurality of light array pads to maximize an intensity of light emitted by the plurality of light array pads without the determined respective temperatures exceeding a threshold temperature.

24. The system of claim 20, wherein the chamber includes a bed and a canopy;
wherein the canopy is operably connected to a cantilever arm extending over the bed.

25. The system of claim 20, wherein the plurality of flexible light array pads include a first set of light emitters configured to emit light in a 630 nm spectrum, a second set of light emitters configured to emit light in a 940 nm spectrum, and a third set of light emitters configured to emit light in a 950 nm spectrum.

26. The system of claim 20, wherein the flexible light array pads have respective sets of windows formed in a patient facing surface;
wherein the plurality of flexible light array pads have respective sets of light emitters positioned in the respective sets of windows.

27. The system of claim 20, wherein the flexible light array pads have respective sets of windows formed in a patient facing surface;
wherein the plurality of flexible light array pads have respective sets of light emitters positioned in the respective sets of windows;
wherein the sets of windows are recessed openings having an inverted pyramidal shape.

28. The system of claim 20, wherein the plurality of flexible light array pads have respective sets of windows formed in a patient facing surface;
wherein the plurality of flexible light array pads have respective sets of light emitters positioned in the respective sets of windows;
wherein the sets of windows are recessed openings have a cone shape.

29. The system of claim 20, wherein the plurality of flexible light array pads have respective sets of windows formed in a patient facing surface;
the plurality of flexible light array pads having respective sets of light emitters positioned in the respective sets of windows;
plurality of flexible light array pads have reflective inserts positioned in the plurality of flexible light array pads.
